SEQUENCE LISTING (46 sequences)

SEQ ID NO: 1 - protein, 9 aa, Artificial Sequence
  tag peptide sequence 1
QKLISEEDL

SEQ ID NO: 2 - protein, 10 aa, Artificial Sequence
  tag peptide sequence 2
EQKLISEEDL

SEQ ID NO: 3 - protein, 14 aa, Artificial Sequence
  tag-linker fusion peptide sequence
EQKLISEEDLGGSG

SEQ ID NO: 4 - protein, 9 aa, Artificial Sequence
  NLS 1
PPPKRPRLD

SEQ ID NO: 5 - protein, 7 aa, Artificial Sequence
  NLS 2
PKKKRKV

SEQ ID NO: 6 - protein, 10 aa, Artificial Sequence
  NLS 3
RADPKKKRKV

SEQ ID NO: 7 - protein, 15 aa, Artificial Sequence
  NLS 4
RPAATKKAGQAKKKK

SEQ ID NO: 8 - protein, 16 aa, Artificial Sequence
  NLS 5
APKKKRKVGIHGVPAA

SEQ ID NO: 9 - protein, 6 aa, Artificial Sequence
  NLS 6
PKKKRK

SEQ ID NO: 10 - protein, 7 aa, Artificial Sequence
  NLS 7
APKKKRK

SEQ ID NO: 11 - protein, 23 aa, Artificial Sequence
  tag-linker-NLS fusion peptide (NAO peptide) sequence
EQKLISEEDLGGSGPPPKRPRLD

SEQ ID NO: 12 - protein, 1404 aa, Artificial Sequence
  HeiCas9 (MFO-Cas9-O)
MEQKLISEEDLGGSGPPPKRPRLDLEDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPPPKRPRLD

SEQ ID NO: 13 - protein, 1406 aa, Artificial Sequence
  MIS-Cas9-S
MEQKLISEEDLGIHGVPAAAPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSAAPKKKRKVD

SEQ ID NO: 14 - protein, 1404 aa, Artificial Sequence
  SMF-Cas9-S
MAPKKKRKVLEEQKLISEEDLGGSGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSAAPKKKRKVD

SEQ ID NO: 15 - protein, 1404 aa, Artificial Sequence
  OMF-Cas9-O
MPPPKRPRLDLEEQKLISEEDLGGSGDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPPPKRPRLD

SEQ ID NO: 16 - DNA, 4215 bp, Artificial Sequence
  HeiCas9 encoding sequence
atggagcagaagctgatcagcgaggaggacctgggaggaagcggaccacctcccaagaggcccaggctggacctcgaggataaaaagtattctattggtttagacatcggcaccaacagcgtgggctgggccgtgatcaccgacgagtacaaggtgcccagcaagaaattcaaggtgctgggcaacaccgacagacacagcatcaagaaaaacctgatcggcgccctgctcttcgactccggcgaaaccgccgaggccaccagactgaagagaaccgccagacggagatacaccagacggaagaatagaatctgctacctgcaggagatcttcagcaacgagatggccaaggtggacgatagcttctttcacagactggaagagagcttcctggtggaagaggacaagaaacacgagagacaccccatcttcggcaacatcgtggacgaggtggcctaccacgagaagtaccccaccatctaccacctgagaaagaaactggtggacagcaccgacaaggccgacctgagactgatctacctggcactggcccacatgatcaagttcagaggccacttcctgatcgagggcgacctgaaccccgacaacagcgacgtggacaagctgttcatccagctggtgcagacctacaaccagctgttcgaagagaaccctatcaacgccagcggcgtggacgccaaggccatcctgagcgccagactcagcaagagcagacggctggagaacctgatcgcccagctgcccggcgagaagaaaaacggcctgttcggcaacctgatcgccctgagcctgggcctgacccccaacttcaagagcaacttcgacctggccgaggacgccaagctgcagctgagcaaggacacctacgacgatgacctggacaacctcctggcccagatcggcgaccagtacgccgacctgttcctcgcagccaagaacctgagcgacgccatcctcctgagcgacatcctcagagtgaacaccgagatcaccaaggctcccctgagcgccagcatgatcaagagatacgacgagcaccatcaggacctgaccctcctgaaggccctcgtgagacaacagctgcccgagaagtacaaggagatcttctttgaccagagcaagaacggctacgccggctacatcgacggaggcgccagtcaggaagagttctacaagttcatcaagcccatcctggagaagatggacggcaccgaagagctgctcgtgaagctgaacagagaggacctgctcagaaagcagagaaccttcgacaacggcagcatcccccaccagatccacctgggcgagctgcacgccatcctgagacggcaggaggacttctaccccttcctgaaggacaacagagagaagattgaaaagatcctgaccttcagaatcccctactatgtgggccccctggccagaggcaacagcagattcgcctggatgaccaggaagagcgaagagacaatcacaccttggaacttcgaagaggtggtcgacaaaggcgccagcgcccagagcttcatcgagagaatgaccaacttcgacaagaacctgcccaacgagaaggtgctgcccaagcacagcctcctgtacgagtacttcaccgtgtacaacgagctgaccaaggtgaagtacgtgaccgagggcatgagaaagcctgcctttctgagtggcgagcagaagaaagccatcgtggacctgctcttcaagaccaacagaaaagtgaccgtgaagcagctgaaggaggactacttcaagaaaatcgagtgcttcgacagcgtggagatcagcggcgtggaggacagattcaacgccagcctgggcacctaccacgacctgctcaagattatcaaagacaaggacttcctggacaacgaagagaacgaggacatcctggaggacatcgtgctgacactgaccctcttcgaggacagagagatgatcgaagagagactgaagacctacgcccacctgttcgatgacaaggtgatgaagcagctgaagagacggagatacaccggctggggcagactgagcagaaagctgatcaacggcatcagagacaagcagagcggcaagaccatcctggacttcctgaagagcgacggcttcgccaacagaaacttcatgcagctgatccacgatgacagcctgaccttcaaggaggacatccagaaagcccaagtgagcgggcagggcgacagcctgcacgagcatatcgccaacctggctggcagccccgccatcaagaaaggcatcctgcagaccgtgaaggtcgtggacgagctggtcaaggtgatgggcagacacaagcccgagaacatcgtgattgagatggccagagagaaccagacaacccagaagggccagaagaacagcagagagagaatgaagagaatcgaagagggcatcaaggagctgggcagccagatcctgaaggagcaccccgtggagaacacccagctgcagaacgagaagctgtacctgtattacctgcagaacggcagagacatgtacgtggaccaggagctggacatcaacagactgagcgattacgacgtggatcacatcgtcccccagagcttcctgaaggatgacagcatcgataacaaggtgctgaccagaagcgacaagaacagaggcaagagcgacaacgtgcccagcgaagaggtcgtgaagaaaatgaagaactactggagacagctcctgaacgccaagctgatcacccagagaaagttcgacaacctgaccaaggccgagagaggcgggctgagcgagctcgacaaagccggcttcatcaagagacagctggtggaaaccagacagatcaccaagcacgtggcccagatcctggacagcagaatgaacaccaagtacgacgagaacgataagctgatcagagaggtgaaggtcatcaccctgaagagcaaactggtgagcgacttcagaaaggacttccagttctacaaggtgagagagatcaataactaccatcacgctcatgacgcctacctgaacgccgtcgtgggcaccgccctgatcaagaaataccccaagctggagagcgagttcgtgtacggcgactacaaggtgtacgacgtgagaaagatgatcgccaagagcgagcaggagatcggcaaggccaccgccaagtacttcttttacagcaacatcatgaacttctttaagaccgagatcaccctggccaacggcgagatcagaaagaggcccctgatcgaaaccaacggcgaaaccggcgagatcgtgtgggacaagggcagagacttcgccaccgtgagaaaggtgctgagcatgccccaggtgaacatcgtgaagaaaaccgaggtgcagaccggaggcttcagcaaggagagcatcctgcccaagagaaacagcgacaagctgatcgccagaaagaaagactgggaccccaagaaatacggaggcttcgacagccccaccgtggcctacagcgtgctggtcgtggccaaggtggagaagggcaagagcaagaaactgaagagcgtgaaggagctcctgggcatcaccatcatggagagatccagcttcgagaagaaccccatcgacttcctggaggccaagggctacaaggaggtgaagaaagacctgattatcaagctgcccaagtacagcctgttcgagctggagaacggcagaaagagaatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgaaatcattgagcagatcagcgagttcagtaagagagtgatcctggctgacgccaacctggacaaggtgctgagcgcctacaacaagcacagagacaagcccatcagagagcaggccgagaacatcattcacctgttcaccctgaccaacctgggcgcacccgcagccttcaagtacttcgacaccacaatcgacagaaagagatacaccagcaccaaggaggtgctggacgccaccctgatccaccagagcatcaccggcctgtacgaaaccagaatcgacctgtcacagcttgggggtgacggatcccctcctcccaagaggcccaggctggactaa

SEQ ID NO: 17 - DNA, 4221 bp, Artificial Sequence
  MIS-Cas9-S encoding sequence
atggagcagaagctgatcagcgaggaggacctgggtatccacggagtcccagcagccgctccaaagaagaagcgtaaggtagataaaaagtattctattggtttagacatcggcaccaacagcgtgggctgggccgtgatcaccgacgagtacaaggtgcccagcaagaaattcaaggtgctgggcaacaccgacagacacagcatcaagaaaaacctgatcggcgccctgctcttcgactccggcgaaaccgccgaggccaccagactgaagagaaccgccagacggagatacaccagacggaagaatagaatctgctacctgcaggagatcttcagcaacgagatggccaaggtggacgatagcttctttcacagactggaagagagcttcctggtggaagaggacaagaaacacgagagacaccccatcttcggcaacatcgtggacgaggtggcctaccacgagaagtaccccaccatctaccacctgagaaagaaactggtggacagcaccgacaaggccgacctgagactgatctacctggcactggcccacatgatcaagttcagaggccacttcctgatcgagggcgacctgaaccccgacaacagcgacgtggacaagctgttcatccagctggtgcagacctacaaccagctgttcgaagagaaccctatcaacgccagcggcgtggacgccaaggccatcctgagcgccagactcagcaagagcagacggctggagaacctgatcgcccagctgcccggcgagaagaaaaacggcctgttcggcaacctgatcgccctgagcctgggcctgacccccaacttcaagagcaacttcgacctggccgaggacgccaagctgcagctgagcaaggacacctacgacgatgacctggacaacctcctggcccagatcggcgaccagtacgccgacctgttcctcgcagccaagaacctgagcgacgccatcctcctgagcgacatcctcagagtgaacaccgagatcaccaaggctcccctgagcgccagcatgatcaagagatacgacgagcaccatcaggacctgaccctcctgaaggccctcgtgagacaacagctgcccgagaagtacaaggagatcttctttgaccagagcaagaacggctacgccggctacatcgacggaggcgccagtcaggaagagttctacaagttcatcaagcccatcctggagaagatggacggcaccgaagagctgctcgtgaagctgaacagagaggacctgctcagaaagcagagaaccttcgacaacggcagcatcccccaccagatccacctgggcgagctgcacgccatcctgagacggcaggaggacttctaccccttcctgaaggacaacagagagaagattgaaaagatcctgaccttcagaatcccctactatgtgggccccctggccagaggcaacagcagattcgcctggatgaccaggaagagcgaagagacaatcacaccttggaacttcgaagaggtggtcgacaaaggcgccagcgcccagagcttcatcgagagaatgaccaacttcgacaagaacctgcccaacgagaaggtgctgcccaagcacagcctcctgtacgagtacttcaccgtgtacaacgagctgaccaaggtgaagtacgtgaccgagggcatgagaaagcctgcctttctgagtggcgagcagaagaaagccatcgtggacctgctcttcaagaccaacagaaaagtgaccgtgaagcagctgaaggaggactacttcaagaaaatcgagtgcttcgacagcgtggagatcagcggcgtggaggacagattcaacgccagcctgggcacctaccacgacctgctcaagattatcaaagacaaggacttcctggacaacgaagagaacgaggacatcctggaggacatcgtgctgacactgaccctcttcgaggacagagagatgatcgaagagagactgaagacctacgcccacctgttcgatgacaaggtgatgaagcagctgaagagacggagatacaccggctggggcagactgagcagaaagctgatcaacggcatcagagacaagcagagcggcaagaccatcctggacttcctgaagagcgacggcttcgccaacagaaacttcatgcagctgatccacgatgacagcctgaccttcaaggaggacatccagaaagcccaagtgagcgggcagggcgacagcctgcacgagcatatcgccaacctggctggcagccccgccatcaagaaaggcatcctgcagaccgtgaaggtcgtggacgagctggtcaaggtgatgggcagacacaagcccgagaacatcgtgattgagatggccagagagaaccagacaacccagaagggccagaagaacagcagagagagaatgaagagaatcgaagagggcatcaaggagctgggcagccagatcctgaaggagcaccccgtggagaacacccagctgcagaacgagaagctgtacctgtattacctgcagaacggcagagacatgtacgtggaccaggagctggacatcaacagactgagcgattacgacgtggatcacatcgtcccccagagcttcctgaaggatgacagcatcgataacaaggtgctgaccagaagcgacaagaacagaggcaagagcgacaacgtgcccagcgaagaggtcgtgaagaaaatgaagaactactggagacagctcctgaacgccaagctgatcacccagagaaagttcgacaacctgaccaaggccgagagaggcgggctgagcgagctcgacaaagccggcttcatcaagagacagctggtggaaaccagacagatcaccaagcacgtggcccagatcctggacagcagaatgaacaccaagtacgacgagaacgataagctgatcagagaggtgaaggtcatcaccctgaagagcaaactggtgagcgacttcagaaaggacttccagttctacaaggtgagagagatcaataactaccatcacgctcatgacgcctacctgaacgccgtcgtgggcaccgccctgatcaagaaataccccaagctggagagcgagttcgtgtacggcgactacaaggtgtacgacgtgagaaagatgatcgccaagagcgagcaggagatcggcaaggccaccgccaagtacttcttttacagcaacatcatgaacttctttaagaccgagatcaccctggccaacggcgagatcagaaagaggcccctgatcgaaaccaacggcgaaaccggcgagatcgtgtgggacaagggcagagacttcgccaccgtgagaaaggtgctgagcatgccccaggtgaacatcgtgaagaaaaccgaggtgcagaccggaggcttcagcaaggagagcatcctgcccaagagaaacagcgacaagctgatcgccagaaagaaagactgggaccccaagaaatacggaggcttcgacagccccaccgtggcctacagcgtgctggtcgtggccaaggtggagaagggcaagagcaagaaactgaagagcgtgaaggagctcctgggcatcaccatcatggagagatccagcttcgagaagaaccccatcgacttcctggaggccaagggctacaaggaggtgaagaaagacctgattatcaagctgcccaagtacagcctgttcgagctggagaacggcagaaagagaatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgaaatcattgagcagatcagcgagttcagtaagagagtgatcctggctgacgccaacctggacaaggtgctgagcgcctacaacaagcacagagacaagcccatcagagagcaggccgagaacatcattcacctgttcaccctgaccaacctgggcgcacccgcagccttcaagtacttcgacaccacaatcgacagaaagagatacaccagcaccaaggaggtgctggacgccaccctgatccaccagagcatcaccggcctgtacgaaaccagaatcgacctgtcacagcttgggggtgacggatccgctgctccaaagaagaagcgtaaggtagattaa

SEQ ID NO: 18 - DNA, 4215 bp, Artificial Sequence
  SMF-Cas9-S encoding sequence
atggctccaaagaagaagcgtaaggtactcgaggagcagaagctgatcagcgaggaggacctgggaggaagcggagataaaaagtattctattggtttagacatcggcaccaacagcgtgggctgggccgtgatcaccgacgagtacaaggtgcccagcaagaaattcaaggtgctgggcaacaccgacagacacagcatcaagaaaaacctgatcggcgccctgctcttcgactccggcgaaaccgccgaggccaccagactgaagagaaccgccagacggagatacaccagacggaagaatagaatctgctacctgcaggagatcttcagcaacgagatggccaaggtggacgatagcttctttcacagactggaagagagcttcctggtggaagaggacaagaaacacgagagacaccccatcttcggcaacatcgtggacgaggtggcctaccacgagaagtaccccaccatctaccacctgagaaagaaactggtggacagcaccgacaaggccgacctgagactgatctacctggcactggcccacatgatcaagttcagaggccacttcctgatcgagggcgacctgaaccccgacaacagcgacgtggacaagctgttcatccagctggtgcagacctacaaccagctgttcgaagagaaccctatcaacgccagcggcgtggacgccaaggccatcctgagcgccagactcagcaagagcagacggctggagaacctgatcgcccagctgcccggcgagaagaaaaacggcctgttcggcaacctgatcgccctgagcctgggcctgacccccaacttcaagagcaacttcgacctggccgaggacgccaagctgcagctgagcaaggacacctacgacgatgacctggacaacctcctggcccagatcggcgaccagtacgccgacctgttcctcgcagccaagaacctgagcgacgccatcctcctgagcgacatcctcagagtgaacaccgagatcaccaaggctcccctgagcgccagcatgatcaagagatacgacgagcaccatcaggacctgaccctcctgaaggccctcgtgagacaacagctgcccgagaagtacaaggagatcttctttgaccagagcaagaacggctacgccggctacatcgacggaggcgccagtcaggaagagttctacaagttcatcaagcccatcctggagaagatggacggcaccgaagagctgctcgtgaagctgaacagagaggacctgctcagaaagcagagaaccttcgacaacggcagcatcccccaccagatccacctgggcgagctgcacgccatcctgagacggcaggaggacttctaccccttcctgaaggacaacagagagaagattgaaaagatcctgaccttcagaatcccctactatgtgggccccctggccagaggcaacagcagattcgcctggatgaccaggaagagcgaagagacaatcacaccttggaacttcgaagaggtggtcgacaaaggcgccagcgcccagagcttcatcgagagaatgaccaacttcgacaagaacctgcccaacgagaaggtgctgcccaagcacagcctcctgtacgagtacttcaccgtgtacaacgagctgaccaaggtgaagtacgtgaccgagggcatgagaaagcctgcctttctgagtggcgagcagaagaaagccatcgtggacctgctcttcaagaccaacagaaaagtgaccgtgaagcagctgaaggaggactacttcaagaaaatcgagtgcttcgacagcgtggagatcagcggcgtggaggacagattcaacgccagcctgggcacctaccacgacctgctcaagattatcaaagacaaggacttcctggacaacgaagagaacgaggacatcctggaggacatcgtgctgacactgaccctcttcgaggacagagagatgatcgaagagagactgaagacctacgcccacctgttcgatgacaaggtgatgaagcagctgaagagacggagatacaccggctggggcagactgagcagaaagctgatcaacggcatcagagacaagcagagcggcaagaccatcctggacttcctgaagagcgacggcttcgccaacagaaacttcatgcagctgatccacgatgacagcctgaccttcaaggaggacatccagaaagcccaagtgagcgggcagggcgacagcctgcacgagcatatcgccaacctggctggcagccccgccatcaagaaaggcatcctgcagaccgtgaaggtcgtggacgagctggtcaaggtgatgggcagacacaagcccgagaacatcgtgattgagatggccagagagaaccagacaacccagaagggccagaagaacagcagagagagaatgaagagaatcgaagagggcatcaaggagctgggcagccagatcctgaaggagcaccccgtggagaacacccagctgcagaacgagaagctgtacctgtattacctgcagaacggcagagacatgtacgtggaccaggagctggacatcaacagactgagcgattacgacgtggatcacatcgtcccccagagcttcctgaaggatgacagcatcgataacaaggtgctgaccagaagcgacaagaacagaggcaagagcgacaacgtgcccagcgaagaggtcgtgaagaaaatgaagaactactggagacagctcctgaacgccaagctgatcacccagagaaagttcgacaacctgaccaaggccgagagaggcgggctgagcgagctcgacaaagccggcttcatcaagagacagctggtggaaaccagacagatcaccaagcacgtggcccagatcctggacagcagaatgaacaccaagtacgacgagaacgataagctgatcagagaggtgaaggtcatcaccctgaagagcaaactggtgagcgacttcagaaaggacttccagttctacaaggtgagagagatcaataactaccatcacgctcatgacgcctacctgaacgccgtcgtgggcaccgccctgatcaagaaataccccaagctggagagcgagttcgtgtacggcgactacaaggtgtacgacgtgagaaagatgatcgccaagagcgagcaggagatcggcaaggccaccgccaagtacttcttttacagcaacatcatgaacttctttaagaccgagatcaccctggccaacggcgagatcagaaagaggcccctgatcgaaaccaacggcgaaaccggcgagatcgtgtgggacaagggcagagacttcgccaccgtgagaaaggtgctgagcatgccccaggtgaacatcgtgaagaaaaccgaggtgcagaccggaggcttcagcaaggagagcatcctgcccaagagaaacagcgacaagctgatcgccagaaagaaagactgggaccccaagaaatacggaggcttcgacagccccaccgtggcctacagcgtgctggtcgtggccaaggtggagaagggcaagagcaagaaactgaagagcgtgaaggagctcctgggcatcaccatcatggagagatccagcttcgagaagaaccccatcgacttcctggaggccaagggctacaaggaggtgaagaaagacctgattatcaagctgcccaagtacagcctgttcgagctggagaacggcagaaagagaatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgaaatcattgagcagatcagcgagttcagtaagagagtgatcctggctgacgccaacctggacaaggtgctgagcgcctacaacaagcacagagacaagcccatcagagagcaggccgagaacatcattcacctgttcaccctgaccaacctgggcgcacccgcagccttcaagtacttcgacaccacaatcgacagaaagagatacaccagcaccaaggaggtgctggacgccaccctgatccaccagagcatcaccggcctgtacgaaaccagaatcgacctgtcacagcttgggggtgacggatccgctgctccaaagaagaagcgtaaggtagattaa

SEQ ID NO: 19 - DNA, 4215 bp, Artificial Sequence
  OMF-Cas9-O encoding sequence
atgccacctcccaagaggcccaggctggacctcgaggagcagaagctgatcagcgaggaggacctgggaggaagcggagataaaaagtattctattggtttagacatcggcaccaacagcgtgggctgggccgtgatcaccgacgagtacaaggtgcccagcaagaaattcaaggtgctgggcaacaccgacagacacagcatcaagaaaaacctgatcggcgccctgctcttcgactccggcgaaaccgccgaggccaccagactgaagagaaccgccagacggagatacaccagacggaagaatagaatctgctacctgcaggagatcttcagcaacgagatggccaaggtggacgatagcttctttcacagactggaagagagcttcctggtggaagaggacaagaaacacgagagacaccccatcttcggcaacatcgtggacgaggtggcctaccacgagaagtaccccaccatctaccacctgagaaagaaactggtggacagcaccgacaaggccgacctgagactgatctacctggcactggcccacatgatcaagttcagaggccacttcctgatcgagggcgacctgaaccccgacaacagcgacgtggacaagctgttcatccagctggtgcagacctacaaccagctgttcgaagagaaccctatcaacgccagcggcgtggacgccaaggccatcctgagcgccagactcagcaagagcagacggctggagaacctgatcgcccagctgcccggcgagaagaaaaacggcctgttcggcaacctgatcgccctgagcctgggcctgacccccaacttcaagagcaacttcgacctggccgaggacgccaagctgcagctgagcaaggacacctacgacgatgacctggacaacctcctggcccagatcggcgaccagtacgccgacctgttcctcgcagccaagaacctgagcgacgccatcctcctgagcgacatcctcagagtgaacaccgagatcaccaaggctcccctgagcgccagcatgatcaagagatacgacgagcaccatcaggacctgaccctcctgaaggccctcgtgagacaacagctgcccgagaagtacaaggagatcttctttgaccagagcaagaacggctacgccggctacatcgacggaggcgccagtcaggaagagttctacaagttcatcaagcccatcctggagaagatggacggcaccgaagagctgctcgtgaagctgaacagagaggacctgctcagaaagcagagaaccttcgacaacggcagcatcccccaccagatccacctgggcgagctgcacgccatcctgagacggcaggaggacttctaccccttcctgaaggacaacagagagaagattgaaaagatcctgaccttcagaatcccctactatgtgggccccctggccagaggcaacagcagattcgcctggatgaccaggaagagcgaagagacaatcacaccttggaacttcgaagaggtggtcgacaaaggcgccagcgcccagagcttcatcgagagaatgaccaacttcgacaagaacctgcccaacgagaaggtgctgcccaagcacagcctcctgtacgagtacttcaccgtgtacaacgagctgaccaaggtgaagtacgtgaccgagggcatgagaaagcctgcctttctgagtggcgagcagaagaaagccatcgtggacctgctcttcaagaccaacagaaaagtgaccgtgaagcagctgaaggaggactacttcaagaaaatcgagtgcttcgacagcgtggagatcagcggcgtggaggacagattcaacgccagcctgggcacctaccacgacctgctcaagattatcaaagacaaggacttcctggacaacgaagagaacgaggacatcctggaggacatcgtgctgacactgaccctcttcgaggacagagagatgatcgaagagagactgaagacctacgcccacctgttcgatgacaaggtgatgaagcagctgaagagacggagatacaccggctggggcagactgagcagaaagctgatcaacggcatcagagacaagcagagcggcaagaccatcctggacttcctgaagagcgacggcttcgccaacagaaacttcatgcagctgatccacgatgacagcctgaccttcaaggaggacatccagaaagcccaagtgagcgggcagggcgacagcctgcacgagcatatcgccaacctggctggcagccccgccatcaagaaaggcatcctgcagaccgtgaaggtcgtggacgagctggtcaaggtgatgggcagacacaagcccgagaacatcgtgattgagatggccagagagaaccagacaacccagaagggccagaagaacagcagagagagaatgaagagaatcgaagagggcatcaaggagctgggcagccagatcctgaaggagcaccccgtggagaacacccagctgcagaacgagaagctgtacctgtattacctgcagaacggcagagacatgtacgtggaccaggagctggacatcaacagactgagcgattacgacgtggatcacatcgtcccccagagcttcctgaaggatgacagcatcgataacaaggtgctgaccagaagcgacaagaacagaggcaagagcgacaacgtgcccagcgaagaggtcgtgaagaaaatgaagaactactggagacagctcctgaacgccaagctgatcacccagagaaagttcgacaacctgaccaaggccgagagaggcgggctgagcgagctcgacaaagccggcttcatcaagagacagctggtggaaaccagacagatcaccaagcacgtggcccagatcctggacagcagaatgaacaccaagtacgacgagaacgataagctgatcagagaggtgaaggtcatcaccctgaagagcaaactggtgagcgacttcagaaaggacttccagttctacaaggtgagagagatcaataactaccatcacgctcatgacgcctacctgaacgccgtcgtgggcaccgccctgatcaagaaataccccaagctggagagcgagttcgtgtacggcgactacaaggtgtacgacgtgagaaagatgatcgccaagagcgagcaggagatcggcaaggccaccgccaagtacttcttttacagcaacatcatgaacttctttaagaccgagatcaccctggccaacggcgagatcagaaagaggcccctgatcgaaaccaacggcgaaaccggcgagatcgtgtgggacaagggcagagacttcgccaccgtgagaaaggtgctgagcatgccccaggtgaacatcgtgaagaaaaccgaggtgcagaccggaggcttcagcaaggagagcatcctgcccaagagaaacagcgacaagctgatcgccagaaagaaagactgggaccccaagaaatacggaggcttcgacagccccaccgtggcctacagcgtgctggtcgtggccaaggtggagaagggcaagagcaagaaactgaagagcgtgaaggagctcctgggcatcaccatcatggagagatccagcttcgagaagaaccccatcgacttcctggaggccaagggctacaaggaggtgaagaaagacctgattatcaagctgcccaagtacagcctgttcgagctggagaacggcagaaagagaatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgaaatcattgagcagatcagcgagttcagtaagagagtgatcctggctgacgccaacctggacaaggtgctgagcgcctacaacaagcacagagacaagcccatcagagagcaggccgagaacatcattcacctgttcaccctgaccaacctgggcgcacccgcagccttcaagtacttcgacaccacaatcgacagaaagagatacaccagcaccaaggaggtgctggacgccaccctgatccaccagagcatcaccggcctgtacgaaaccagaatcgacctgtcacagcttgggggtgacggatcccctcctcccaagaggcccaggctggactaa

SEQ ID NO: 20 - protein, 8 aa, Artificial Sequence
  Internal linker
GIHGVPAA

SEQ ID NO: 21 - protein, 4 aa, Artificial Sequence
  flexible linker
GGSG

SEQ ID NO: 22 - protein, 1371 aa, Artificial Sequence
  partial sequence of Cas9
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSAA

SEQ ID NO: 23 - DNA, 76 bp, Artificial Sequence
  myc-Cas9 forward
aatttaccggtcaaacatggagcagaagctgatcagcgaggaggacctgatggccccaaagaagaagcggaaggtc

SEQ ID NO: 24 - DNA, 119 bp, Artificial Sequence
  heiCas9 forward
aatttaccggtttaccatggagcagaagctgatcagcgaggaggacctgggaggaagcggaccacctcccaagaggcccaggctggacctcgaggataaaaagtattctattggtttag

SEQ ID NO: 25 - DNA, 23 bp, Artificial Sequence
  pCS2+ backbone forward
gcctctagaactatagtgagtcg

SEQ ID NO: 26 - DNA, 44 bp, Artificial Sequence
  hei-tag forward
cttgttctttttgcaggatcccatttaccatggagcagaagctg

SEQ ID NO: 27 - DNA, 33 bp, Artificial Sequence
  Gam Mu-APOBEC1-Cas9n fragment forward
gacctcgaggctaaaccagcaaaacgtatcaag

SEQ ID NO: 28 - DNA, 20 bp, Artificial Sequence
  Cas9n-UGI fragment forward
gacacttctcaaggccctag

SEQ ID NO: 29 - DNA, 19 bp, Artificial Sequence
  2xUGI-oNLS forward
cagcttgggggtgactctg

SEQ ID NO: 30 - DNA, 66 bp, Artificial Sequence
  heiCas9 reverse
aattttctagattagtccagcctgggcctcttgggaggaggggatccgtcacccccaagctgtgac

SEQ ID NO: 31 - DNA, 38 bp, Artificial Sequence
  myc-Cas9 reverse
aattttctagattactttttcttttttgcctggccggc

SEQ ID NO: 32 - DNA, 24 bp, Artificial Sequence
  pCS2+ backbone reverse
atgggatcctgcaaaaagaacaag

SEQ ID NO: 33 - DNA, 28 bp, Artificial Sequence
  hei-tag reverse
gctggtttagcctcgaggtccagcctgg

SEQ ID NO: 34 - DNA, 20 bp, Artificial Sequence
  Gam Mu-APOBEC1-Cas9n fragment reverse
ctagggccttgagaagtgtc

SEQ ID NO: 35 - DNA, 19 bp, Artificial Sequence
  Cas9n-UGI fragment reverse
cagagtcacccccaagctg

SEQ ID NO: 36 - DNA, 69 bp, Artificial Sequence
  2xUGI-oNLS reverse
cgactcactatagttctagaggcttagtccagcctgggcctcttgggagggggagaaccaccagagagc

SEQ ID NO: 37 - DNA, 29 bp, Artificial Sequence
  Oca2 fwd
gttaaaacagtttcttaaaaagaacagga

SEQ ID NO: 38 - DNA, 26 bp, Artificial Sequence
  Oca2 rev
agcagaagaaatgactcaacattttg

SEQ ID NO: 39 - DNA, 23 bp, Artificial Sequence
  MmPrx exon 6 crRNA
tcgtatccagacaccgtcccggg

SEQ ID NO: 40 - DNA, 20 bp, Artificial Sequence
  MmPrx exon 6 fwd
gagacactcactccagaccc

SEQ ID NO: 41 - DNA, 20 bp, Artificial Sequence
  MmPrx exon 6 rev
actcagtaacccaacagcca

SEQ ID NO: 42 - DNA, 25 bp, Artificial Sequence
  Fig. 4 Upper
ggaaacccaggtggccattgcagga

SEQ ID NO: 43 - protein, 8 aa, Artificial Sequence
  Fig. 4 middle
ETQVAIAG

SEQ ID NO: 44 - DNA, 12 bp, Artificial Sequence
  Fig. 4 lower
gaaatttaggtg

SEQ ID NO: 45 - protein, 8 aa, Artificial Sequence
  NLS8
APKKKRKV

SEQ ID NO: 46 - protein, 4 aa, Artificial Sequence
  Internal linker 2
VPAA

(19) United States
(12) Patent Application Publication
(10) Pub. No.: US 2024/0200045 A1
(43) Pub. Date: Jun. 20, 2024

(54) NUCLEASE WITH IMPROVED TARGETING ACTIVITY

(71) Applicant: Universität Heidelberg, Heidelberg (DE)

(72) Inventors: Jose Arturo Gutierrez Triana, Floridablanca- Santander (CO); Joachim Wittbrodt, Bammental (DE); Thomas Thumberger, Neckargemünd (DE); Tinatini Tavhelidse-Suck, Wiesloch (DE)

(21) Appl. No.: 18/553,700

(22) PCT Filed: Mar. 29, 2022
(86) PCT No.: PCT/EP2022/058214

§ 371 Date: Oct. 2, 2023

(30) Foreign Application Priority Data
Mar. 30, 2021 (EP) 21166099.8

(51) Int. Cl. C12N 9/22; A61K 38/00

(52) U.S. Cl. CPC C12N 9/22; A61K 38/00; C07K 2319/09; C07K 2319/41; C12N 2310/20

(57) ABSTRACT

The present invention relates to a polynucleotide encoding a nuclear polypeptide comprising a cargo polypeptide and an N-terminal activity-optimizing peptide (NAO peptide), wherein said NAO peptide comprises (i) a tag peptide, (ii) a linker peptide; and (iii) a nuclear localization sequence (NLS) peptide wherein said linker peptide comprises at least three small amino acids independently selected from glycine, alanine, leucine, serine, aspartate, asparagine, threonine, phenylalanine, glutamate, glutamine, histidine, arginine, lysine, valine, isoleucine, and proline, more preferably from glycine, alanine, proline, serine, valine, and threonine; and wherein said tag peptide is fused at its C-terminus to the linker peptide; and to vectors, polypeptides, host cells, and methods related thereto.

The present invention relates to a polynucleotide encoding a nuclear polypeptide comprising a cargo polypeptide and an N-terminal activity-optimizing peptide (NAO peptide), wherein said NAO peptide comprises (i) a tag peptide, (ii) a linker peptide; and (iii) a nuclear localization sequence (NLS) peptide, wherein said linker peptide comprises at least three small amino acids independently selected from glycine, alanine, leucine, serine, aspartate, asparagine, threonine, phenylalanine, glutamate, glutamine, histidine, arginine, lysine, valine, isoleucine, and proline, more preferably from glycine, alanine, proline, serine, valine, and threonine; and wherein said tag peptide is fused at its C-terminus to the linker peptide. The present invention further relates to vectors, polypeptides, host cells, and methods related to said polynucleotide.

Precise, targeted modification of the genome is the key prerequisite for addressing the function of the genome in basic research as well as in translational approaches. With the adaptation of the bacterial CRISPR/Cas9 system, genome editing approaches are applicable to an almost unlimited range of model systems. Its RNA guided endonuclease activity has been experimentally employed for sequence-specific editing in organisms ranging from bacteria to plants and animals. The targeting mediated by Cas9 and its variants promises a manipulation of genomic loci with unprecedented precision (Wang, 2016). While active in any species tested so far, efficiencies still leave a lot of room for improvement. A high efficiency will be essential for precise target sequence modification with limited off target effects as ultimately required for therapeutic interventions. Assessing the efficiency of the intervention requires a quantitative readout that ideally directly addresses the target resulting in an apparent phenotype.

For reaching its target, the bacterial Cas9 needs to be efficiently shuttled into the nucleus. This has been attempted by N- and/or C-terminally fusing the bacterial Cas9 protein with nuclear localization signals (NLSs) (Cong, 2013). To facilitate detection of the Cas9 protein and additionally evaluate the efficacy of the approach, Cas9 variants have been tagged with different epitopes (e.g. in Zhang, 2014, US 2014/273226 A1, and WO 2014/204727 A1).

Nonetheless, efficiency of genome editing in most cases is far from optimal. There is, thus, a need in the art for improved means and methods for genome editing. It is therefore an objective of the present invention to provide means and methods to comply with the aforementioned needs, avoiding at least in part the disadvantages of the prior art. This problem is solved by compounds, methods, and uses of the present invention. Embodiments, which might be realized in an isolated fashion or in any arbitrary combination, are listed in the dependent claims.

In accordance, the present invention relates to a polynucleotide encoding a nuclear polypeptide comprising a cargo polypeptide and an N-terminal activity-optimizing peptide (NAO peptide), wherein said NAO peptide comprises
(i) a tag peptide;
(ii) a linker peptide; and
(iii) a nuclear localization sequence (NLS) peptide
wherein said linker peptide comprises at least three small amino acids independently selected from glycine, alanine, leucine, serine, aspartate, asparagine, threonine, phenylalanine, glutamate, glutamine, histidine, arginine, lysine, valine, isoleucine, and proline, preferably from glycine, alanine, proline, serine, valine, and threonine; and wherein preferably said tag peptide is fused at its C-terminus to the linker peptide.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” may comprise any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s). In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or activity. Thus, the term includes sub-parts generated by actual fragmentation of a biological macromolecule, but also sub-parts derived from the respective biological macromolecule in an abstract manner, e.g. in silico. Thus, as used herein, an Fc or Fab fragment, but also e.g. a single-chain antibody, a bispecific antibody, and a nanobody may be referred to as fragments of an immunoglobulin.

Unless specifically indicated otherwise herein, the compounds specified, in particular the nuclear polypeptides, may be comprised in larger structures, e.g. may be covalently or non-covalently linked to carrier molecules, retardants, and other excipients. In particular, polypeptides as specified may be comprised in nuclear polypeptides comprising further peptides and/or may be part of complexes comprising the nuclear polypeptide, e.g. homo- or heteromultimers of the nuclear polypeptides, optionally including one or more further polypeptide(s), such as dimers, tetramers, but also including e.g. virus capsids; affinity complexes, e.g. antibody/nuclear polypeptide complexes; and/or liposomes or micelles comprising a nuclear polypeptide or a polynucleotide referred to herein.

The term “polynucleotide” is known to the skilled person. As used herein, the term includes nucleic acid molecules comprising or consisting of a nucleic acid sequence or nucleic acid sequences as specified herein, having the activity of encoding a nuclear polypeptide as specified herein below. The polynucleotide shall be provided, preferably, either as an isolated polynucleotide (i.e. isolated from its natural context) or in genetically modified form. The polynucleotide, preferably, is DNA, including cDNA, or is RNA. The term encompasses single as well as double stranded polynucleotides. Preferably, the polynucleotide is a chimeric molecule, i.e., preferably, comprises at least one nucleic acid sequence, preferably of at least 20 bp, more preferably at least 100 bp, heterologous to the residual nucleic acid sequences. Moreover, preferably, comprised are also chemically modified polynucleotides including naturally occurring modified polynucleotides such as glycosylated or methylated polynucleotides or artificial modified ones such as biotinylated polynucleotides. In view of the description herein below, the polynucleotide preferably comprises, more preferably consists of, the nucleic acid sequence of SEQ ID NO: 16 or a nucleic acid sequence at least 60% identical to SEQ ID NO: 16, of SEQ ID NO: 17 or a nucleic acid sequence at least 60% identical to SEQ ID NO:17; SEQ ID NO:18 or a nucleic acid sequence at least 60% identical to SEQ ID NO:18, or of SEQ ID NO: 19 or a nucleic acid sequence at least 60% identical to SEQ ID NO: 19. As is understood by the skilled person, the term “at least 60% identical” includes at least 70% identity, at least 80% identity, at least 90% identity, at least 95% identity, at least 98% identity, at least 99% identity, as well as 100% identity.

As used herein, the term polynucleotide, preferably, includes variants of the specifically indicated polynucleotides, said variants having the activity of encoding a nuclear polypeptide as specified herein below. More preferably, the term polynucleotide relates to the specific polynucleotides indicated. It is to be understood, however, that a polypeptide having a specific amino acid sequence may be also encoded by a variety of polynucleotides, due to the degeneration of the genetic code. The skilled person knows how to select a polynucleotide encoding a polypeptide having a specific amino acid sequence and also knows how to optimize the codons used in the polynucleotide according to the codon usage of the organism used for expressing said polynucleotide. Thus, the term “polynucleotide variant”, as used herein, relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide. Moreover, it is to be understood that a polynucleotide variant as referred to in accordance with the present invention shall have a nucleic acid sequence which differs due to at least one nucleotide substitution, deletion and/or addition from the specifically indicated polynucleotide. Preferably, said polynucleotide variant is an ortholog, a paralog or another homolog of the specific polynucleotide. Also preferably, said polynucleotide variant is a naturally occurring allele of the specific polynucleotide. Polynucleotide variants also encompass polynucleotides comprising a nucleic acid sequence which is capable of hybridizing to the aforementioned specific polynucleotides, preferably, under stringent hybridization conditions. These stringent conditions are known to the skilled worker and can be found in Current Protocols in Molecular Biology, John Wiley & Sons, N. Y. (1989), 6.3.1-6.3.6. A preferred example for stringent hybridization conditions are hybridization conditions in 6× sodium chloride/sodium citrate (=SSC) at approximately 45° C., followed by one or more wash steps in 0.2×SSC, 0.1% SDS at 50 to 65° C. The skilled worker knows that these hybridization conditions differ depending on the type of nucleic acid and, for example when organic solvents are present, with regard to the temperature and concentration of the buffer. For example, under “standard hybridization conditions” the temperature differs depending on the type of nucleic acid between 42° C. and 58° C. in aqueous buffer with a concentration of 0.1× to 5×SSC (pH 7.2). If organic solvent is present in the abovementioned buffer, for example 50% formamide, the temperature under standard conditions is approximately 42° C. The hybridization conditions for DNA:DNA hybrids are preferably for example 0.1×SSC and 20° C. to 45° C., preferably between 30° C. and 45° C. The hybridization conditions for DNA:RNA hybrids are preferably, for example, 0.1×SSC and 30° C. to 55° C., preferably between 45° C. and 55° C. The abovementioned hybridization temperatures are determined for example for a nucleic acid with approximately 100 bp (=base pairs) in length and a G+C content of 50% in the absence of formamide. The skilled worker knows how to determine the hybridization conditions required by referring to textbooks such as the textbook mentioned above, or the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford. Alternatively, polynucleotide variants are obtainable by PCR-based techniques such as mixed oligonucleotide primer-based amplification of DNA, i.e. using degenerated primers against conserved domains of a polypeptide as specified herein. Conserved domains of a polypeptide may be identified by a sequence comparison of the nucleic acid sequence of the polynucleotide or the amino acid sequence of the polypeptide of the present invention with sequences of other organisms. As a template, DNA or cDNA from bacteria, fungi, plants or, preferably, from animals may be used. Further, variants include polynucleotides comprising nucleic acid sequences which are at least 70%, at least 75%, at least 80%, at least 85%, at least 90%, at least 95%, at least 98% or at least 99% identical to the specifically indicated nucleic acid sequences. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are, preferably, calculated over the entire amino acid or nucleic acid sequence region, preferably as specified herein above. The sequence identity values recited above in percent (%) are to be determined for polynucleotides, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the specifically indicated nucleic acid sequences is also encompassed as a variant polynucleotide. The fragment shall still encode a nuclear polypeptide which still has the activity as specified. Accordingly, the nuclear polypeptide encoded may comprise or consist of the domains of the nuclear polypeptide as specified herein conferring the indicated biological activity. A fragment as meant herein, preferably, comprises at least 50, at least 100, at least 250 or at least 500 consecutive nucleotides of any one of the specific nucleic acid sequences or encodes an amino acid sequence comprising at least 20, at least 30, at least 50, at least 80, at least 100 or at least 150 consecutive amino acids of any one of the specific amino acid sequences.

The polynucleotides of the present invention either consist of, essentially consist of, or comprise the aforementioned nucleic acid sequences. Thus, they may contain further nucleic acid sequences as well. Preferably, in particular in case the polynucleotide is a DNA, the polynucleotide further comprises at least one expression control sequence operatively linked to the sequence encoding the nuclear polypeptide, allowing expression of the nuclear polypeptide in eukaryotic and/or prokaryotic cells or isolated fractions thereof. Expression of said polynucleotide comprises transcription of the polynucleotide or parts thereof, preferably into a translatable mRNA; said transcription may be accomplished in a host cell or in vitro. Appropriate host cells and in vitro transcription systems are known in the art, e.g. the T7 in vitro transcription system. Regulatory elements ensuring expression in eukaryotic cells, preferably mammalian cells, are also well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Possible regulatory elements permitting expression in prokaryotic and/or eukaryotic host cells are known in the art. Examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression vector encompassed by the present invention. Such inducible vectors may comprise tet or lac operator sequences or sequences inducible by heat shock, tamoxifen, trans-tamoxifen, mifepristone, or other chemical or environmental factors. Besides elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as the pCS2 or pCS2+ vectors as used herein in the Examples, Okayama-Berg cDNA expression vector pcDVI (Pharmacia), pBluescript (Stratagene), pCDM8, pRc/CMV, pcDNA1, pcDNA3 (Invitrogen) or pSPORT1 (GIBCO BRL). Also preferably, in particular in case the polynucleotide is an RNA, preferably an mRNA, the polynucleotide further comprises at least one sequence mediating and/or enhancing translation initiation, preferably a Kozak sequence, a ribosome binding site, and/or an internal ribosome entry site (IRES), and/or one or more sequence(s) or other structural element(s) stabilizing the polynucleotide, in particular a 5′-Cap and/or a 3′-polyA tail.

The term “polypeptide”, as used herein, refers to a molecule consisting of several, typically more than 25 amino acids that are covalently linked to each other by peptide bonds. Molecules consisting of up to 25 amino acids covalently linked by peptide bonds are usually considered to be “peptides”. Preferably, the polypeptide comprises of from 50 to 1000, more preferably of from 75 to 1000, still more preferably of from 100 to 500, most preferably of from 110 to 400 amino acids. Preferably, the polypeptide is comprised in a polypeptide complex.

The term “nuclear polypeptide” relates to a polypeptide comprising the components as specified, i.e. a cargo polypeptide and an N-terminal activity-optimizing peptide (NAO peptide), both as specified herein below. The nuclear polypeptide has the activity of being transported into the nucleus of a cell of a eukaryote. Preferably, the nuclear polypeptide is a non-covalent complex comprising the NAO peptide and the cargo polypeptide; in such case, the NAO peptide and the cargo polypeptide may further comprise partners of an affinity pair, respectively, e.g. biotin and streptavidin; appropriate affinity pairs are known in the art. Preferred are affinity pairs having a dissociation constant K_{d} of at most 10⁻⁷ M, more preferably at most 10⁻⁸ M, most preferably at most 10⁻⁹ M. More preferably, in the nuclear polypeptide, the NAO peptide is covalently bound to the cargo peptide. Most preferably, in the nuclear polypeptide, the NAO peptide is fused to the cargo polypeptide, i.e. the NAO peptide and the cargo polypeptide are comprised of a continuous chain of peptide bonds, which can, preferably, be expressed from a single open reading frame of a polynucleotide. Said fusion may be direct, i.e. the last amino acid of the NAO peptide may be followed by the first amino acid of the nuclear peptide; or said fusion may be indirect, i.e. there may be additional amino acids intervening the last amino acid of the NAO peptide and the first amino acid corresponding to the cargo polypeptide. Preferably, fusion of the NAO peptide to the cargo polypeptide is direct or indirect with at most 25 amino acids, preferably at most 10 amino acids, still more preferably at most 5 amino acids, even more preferably at most 2 amino acids, intervening the NAO peptide and the cargo polypeptide.

As indicated, the NAO peptide is an N-terminal peptide in the nuclear polypeptide, i.e. it is located N-terminal relative to the cargo polypeptide; i.e. the NAO peptide is fused at its C-terminus to the cargo polypeptide in the nuclear polypeptide. Also, in the NAO peptide, the tag peptide preferably is fused at its C-terminus to the linker peptide. The expression “A is fused at its C-terminus to B” is understood by the skilled person to relate to a fusion in which the C-terminal amino acid of A is N-terminal (upstream) of the N-terminal amino acid of B.

It is also understood by the skilled person that it is determined whether additional amino acids intervening between two components of the nuclear polypeptide are present by first determining the last amino acid of the N-terminal component and the first amino acid of the C-terminal component, and then determining whether there are amino acids intervening the last amino acid of the N-terminal component and the first amino acid of the C-terminal component. The last amino acid of the N-terminal component is preferably determined by determining the most C-terminal sequence of 5, preferably 10, continuous amino acids identical to the N-terminal component, and defining the most C-terminal amino acid of said sequence to be the last amino acid of the N-terminal component. In accordance, the first amino acid of the C-terminal component is preferably determined by determining the most N-terminal sequence of 5, preferably 10, continuous amino acids identical to the C-terminal component, and defining the most N-terminal amino acid of said sequence to be the first amino acid of the N-terminal component.

Preferably, the nuclear polypeptide further comprises an NLS peptide C-terminal of the cargo polypeptide, wherein the fusion between the C-terminal NLS peptide preferably is direct or indirect in analogy to the specification herein above for the NAO peptide, more preferably is direct. Also in accordance with the above, the cargo polypeptide in the nuclear polypeptide may lack a number of C-terminal amino acids, provided that the activity of the cargo polypeptide is essentially maintained. Thus, preferably, in the nuclear polypeptide the C-terminal 25 amino acids, more preferably the C-terminal 10 amino acids, still more preferably the C-terminal five amino acids, even more preferably the C-terminal two amino acids, of the cargo polypeptide may be lacking. Preferably, the C-terminal NLS peptide comprises, preferably consists of, an NLS peptide as specified herein below for the NLS peptide comprised in the NAO peptide. More preferably, the C-terminal NLS and at least one NLS of the NAO peptide are essentially identical, preferably are identical. Preferably, the C-terminal NLS has the same amino acid sequence as the NLS comprised in the NAO peptide, preferably the amino acid sequence of SEQ ID NO:4. Also preferably, the nuclear polypeptide comprises, preferably consists of, the sequence of SEQ ID NO:12, 13, 14, or 15, or is a polypeptide variant thereof. More preferably, the nuclear polypeptide comprises, preferably consists of, the sequence of SEQ ID NO: 12, 13, 14, or 15, more preferably of SEQ ID NO: 12.

The term “cargo polypeptide”, as used herein, includes each and every polypeptide being (i) an enzyme having at least one of its substrates located in the nucleus of a eukaryotic cell; (ii) a polypeptide having its physiological location in the nucleus of a eukaryotic cell; (iii) any polypeptide for which nuclear localization is desired or desirable; and/or (iv) any polypeptide variant or fragment thereof, wherein a variant or fragment of a polypeptide of (i) or (ii) preferably has the indicated activity and/or amino acid sequence. The eukaryotic cell preferably is a cell of a eukaryote as specified herein below. As will be understood by the skilled person, the cargo polypeptide in the nuclear polypeptide as specified herein is transported with high efficiency into the nucleus of eukaryotic cells, so the cargo polypeptide may be any polypeptide for which transport into the nucleus is desirable to the skilled person. Thus, the cargo polypeptide may be a cargo polypeptide, which may have one or more NLS sequences, for which it is desirable that transport into the nucleus is enhanced. The cargo polypeptide may also be a non-cargo polypeptide for which redirection to the nucleus is desirable. The cargo polypeptide may also be a heterologous polypeptide, e.g. a non-eukaryotic polypeptide, preferably a bacterial polypeptide, for which location in the nucleus is desirable. Thus, preferably, the cargo polypeptide is a polypeptide of a microorganism, such as of a virus or a bacterium, e.g. a viral capsid polypeptide, a viral regulatory polypeptide, a viral or bacterial enzyme; or is a polypeptide of a eukaryotic cell, such as a gene regulatory polypeptide, e.g. a transcription factor, a transcriptional activator or repressor, or a DNA or RNA processing enzyme. Preferably, the cargo polypeptide is an enzyme having DNA and/or RNA as a substrate, more preferably is a nuclease or a base modifying enzyme, preferably a DNase, an RNase, a deaminase, a methylase, an acetylase, a transposase, a restriction enzyme (e.g. meganuclease), a recombinase, or a DNA-interacting protein (e.g. a transcription activator-like effector nuclease (TALEN) or a Zinc finger protein). Preferably, the cargo polypeptide is a clustered regularly interspaced short palindromic repeats (CRISPR) associated (Cas) polypeptide. Cas polypeptides having nuclease activity are known in the art, as are derivatives thereof having a modified enzymatic activity, e.g. as a nickase or as a deaminase. Preferably, the Cas polypeptide is a Cas9 polypeptide, more preferably a Cas polypeptide from Streptococcus pyogenes or a derivative thereof, more preferably a humanized derivative thereof. Preferably, the Cas polypeptide is a Cas endonuclease. Preferably the Cas polypeptide has the amino acid sequence of SEQ ID NO:22 or a polypeptide variant thereof having the indicated activity. As the skilled person understands, in many polypeptides the first N-terminal amino acids, preferably the first 25 amino acids, more preferably the first ten amino acids, still more preferably the first five amino acids, even more preferably the first two, N-terminal amino acids are dispensable for activity of the polypeptide and can be omitted in construction of a nuclear polypeptide essentially without loss of activity.

The term “N-terminal activity-optimizing peptide”, also referred to as “NAO peptide” herein, relates to any peptide comprising the specified components, i.e. a tag peptide, a linker peptide, and an NLS peptide, all as specified herein below, wherein tag peptide preferably is fused at its C-terminus to the linker peptide. The fusion between the components of the NAO peptide is independently selected from a direct fusion and an indirect fusion, both as specified herein above. In accordance, the NAO peptide may comprise up to 25, preferably up to 10, more preferably up to 5, still more preferably up to 2, most preferably one, amino acid intervening any two of the aforesaid components. More preferably, the fusion between at least two components of the NAO is direct, most preferably the fusions between any two of the components of the NAO peptide are direct. Preferably, the sequence of the elements of the NAO peptide is tag peptide/linker peptide/NLS peptide or NLS peptide/tag peptide/linker peptide, preferably is tag peptide/linker peptide/NLS peptide. Preferably, the NAO peptide comprises, preferably consists of, the amino acid sequence EQKLISEEDLGGSGPPPKRPRLD (SEQ ID NO:11).

The term “tag peptide”, as used herein, relates to any tag peptide deemed appropriate by the skilled person, preferably a peptide having a net negative charge and comprising of from 5 to 25, preferably of from 8 to 20, more preferably of from 9 to 15, more preferably about 10, most preferably 10, amino acids. Preferably, the tag peptide has a net charge of at least -1, more preferably of at least -2, still more preferably of -3 under standard conditions. Preferably, the tag peptide comprises at least four negatively charged amino acids within a sequence of ten continuous amino acids and/or comprises at least three glutamate residues within a sequence of ten continuous amino acids. More preferably, the tag peptide comprises four negatively charged amino acids within a sequence of ten continuous amino acids and/or comprises three glutamate residues within a sequence of ten continuous amino acids. As the skilled person understands, net charge of a peptide is determined under standard conditions, in particular at a pH of 7. Preferably, the tag peptide comprises a c-myc tag peptide, more preferably comprises, still more preferably consists of, the sequence QKLISEEDL (SEQ ID NO:1) or, more preferably, EQKLISEEDL (SEQ ID NO:2). Also preferably, the tag peptide/linker peptide fusion comprises, preferably consists of, the sequence EQKLISEEDLGGSG (SEQ ID NO:3).

The term “linker peptide” is, in principle, understood by the skilled person to relate to each and every peptide providing a connection between two (poly)peptides via peptide bonds and which may, in principle, have any arbitrary amino acid sequence. As used herein, the linker peptide is a peptide as specified herein above, i.e. comprises at most 25 amino acid and further comprises at least three small amino acids independently selected from glycine, alanine, leucine, serine, aspartate, asparagine, threonine, phenylalanine, glutamate, glutamine, histidine, arginine, lysine, valine, isoleucine, and proline, more preferably from glycine, alanine, proline, serine, valine, and threonine. Preferably, the linker peptide comprises said small amino acids within a sequence of at most 10 amino acids, preferably at most 5 amino acids, more preferably comprises at least three consecutive small amino acids. Preferably, the small amino acids are independently selected from glycine, alanine, leucine, serine, aspartate, asparagine, threonine, phenylalanine, glutamate, glutamine, histidine, arginine, lysine, valine, isoleucine, and proline, more preferably from glycine, alanine, proline, serine, valine, and threonine, more preferably from glycine, alanine, proline, and serine, most preferably from glycine and serine. Preferably, the linker peptide consists of from 3 to 25 amino acids, more preferably of from 3 to 10 amino acids, still more preferably of from 3 to 5 amino acids, most preferably of four amino acids. Preferably, the linker comprises, preferably consists of, the sequence GIHGVPAA (SEQ ID NO:20). Preferably, the linker peptide comprises, more preferably consists of, at least three, preferably at least four, consecutive non-charged amino acids. Preferably, the linker peptide is a flexible linker peptide comprising the amino acid sequence GG, GS, and/or SG, more preferably comprising, preferably consisting of, the amino acid sequence GGS and/or GSG, most preferably the amino acid sequence GGSG (SEQ ID NO:21).

The term “nuclear localization sequence”, also referred to as “NLS”, is known to the skilled person, as are sequence features of NLSs. Preferably, the NLS is a short sequence of from 3 to 25 amino acids, preferably of from 5 to 20 amino acids, more preferably of from 7 to 15 amino acids, comprising at least 3 positively charged amino acids, preferably lysine and/or arginine residues. As is understood by the skilled person, the term “NLS peptide” relates to a peptide consisting of an NLS. Preferably, the NLS peptide comprises, preferably consists of, the amino acid sequence PPPKRPRLD (SEQ ID NO:4), PKKKRKV (SEQ ID NO:5), RADPKKKRKV (SEQ ID NO:6), RPAATKKAGQAKKKK (SEQ ID NO: 7), APKKKRKVGIHGVPAA (SEQ ID NO:8), PKKKRK (SEQ ID NO:9), APKKKRK (SEQ ID NO:10), or APKKKRKV (SEQ ID NO: 45); more preferably, the NLS peptide comprises, preferably consists of, the amino acid sequence PPPKRPRLD (SEQ ID NO:4) or PKKKRKV (SEQ ID NO:5), most preferably PPPKRPRLD (SEQ ID NO:4).

Advantageously, it was found in the work underlying the present invention that an NAO peptide as specified improves nuclear import of polypeptide, even if these are not natural cargo polypeptides, e.g. polypeptides derived from bacteria. In accordance, efficacy of e.g. genome modification by a Cas nuclease could be drastically improved.

The definitions made above apply mutatis mutandis to the following. Additional definitions and explanations made further below also apply for all embodiments described in this specification mutatis mutandis.

The present invention also relates to a vector comprising a polynucleotide of the present invention.

The term “vector”, preferably, encompasses phage, plasmid, and viral vectors, as well artificial chromosomes, such as bacterial or yeast artificial chromosomes. Moreover, the term also relates to targeting constructs which allow for random or site- directed integration of the targeting construct into genomic DNA. Such target constructs, preferably, comprise DNA of sufficient length for either homologous or heterologous recombination as described in detail below. The vector encompassing the polynucleotide of the present invention, preferably, further comprises selectable markers for propagation and/or selection in a host. The vector may be incorporated into a host cell by various techniques well known in the art. For example, a plasmid vector can be introduced into a cell a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, it may be packaged in vitro using an appropriate packaging cell line prior to application to host cells. Retroviral vectors may be replication competent or replication defective. In the latter case, viral propagation generally will occur only in complementing host/cells.

Preferably, in the vector of the invention the polynucleotide is operatively linked to expression control sequences allowing expression in prokaryotic and/or eukaryotic cells or isolated fractions thereof, preferably as specified herein above. Thus, preferably, the vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

The present invention also relates to a polypeptide encoded by a polynucleotide of the present invention, i.e. to a nuclear polypeptide comprising a cargo polypeptide and an N-terminal activity-optimizing peptide (NAO peptide), wherein said NAO peptide comprises (i) a tag peptide, (ii) a linker peptide; and (iii) a nuclear localization sequence (NLS) peptide, wherein said linker peptide comprises at least three small amino acids independently selected from glycine, alanine, leucine, serine, aspartate, asparagine, threonine, phenylalanine, glutamate, glutamine, histidine, arginine, lysine, valine, isoleucine, and proline more preferably from glycine, alanine, proline, serine, valine, and threonine; and wherein said tag peptide is preferably fused at its C-terminus to the linker peptide, as specified herein above.

The present invention also relates to a host cell comprising a polynucleotide of the present invention, a vector of the present invention, and/or a polypeptide of the present invention.

As used herein, the term “host cell” relates to any cell capable of receiving and, preferably maintaining, the polynucleotide and/or the vector of the present invention. More preferably, the host cell is capable of expressing a nuclear polypeptide as specified herein encoded on said polynucleotide and/or vector. Preferably, the cell is a bacterial cell, more preferably a cell of a common laboratory bacterial strain known in the art, most preferably an Escherichia strain, in particular an E. coli strain. Also preferably, the host cell is a cell of an eukaryote as specified elsewhere herein. Thus, the host cell may be a cell of a non-animal eukaryote as specified elsewhere herein, or a cell of an animal eukaryote, preferably of a subject as specified elsewhere herein. Preferably, the host cell is a chordate cell, more preferably a vertebrate cell, more preferably a human, an amphibian, or a teleost cell, most preferably a human cell. Preferably, the host cell is a cultured cell.

The present invention further relates to a kit comprising a polynucleotide of the present invention, a vector of the present invention, and/or a nuclear polypeptide of the present invention in a housing.

The term “kit”, as used herein, refers to a collection of the aforementioned compounds, means or reagents of the present invention which may or may not be packaged together. The components of the kit may be comprised by separate vials (i.e. as a kit of separate parts) within the housing or provided in a single vial. As used herein, the term “housing” relates to a casing comprising the components as specified, preferably enabling transport, preferably translocation, of the compound or compounds. Moreover, it is to be understood that the kit of the present invention, preferably, is to be used for practicing the methods referred to herein above. It is, preferably, envisaged that all components are provided in a ready-to-use manner for practicing the methods referred to above. Further, the kit, preferably, contains instructions for carrying out said methods. The instructions can be provided by a user's manual in paper or electronic form. In addition, the manual may comprise instructions for administration and/or dosage instructions for carrying out the aforementioned methods using the kit of the present invention.

Preferably, the kit further comprises a means mediating entry of the nuclear polypeptide, the polynucleotide, and/or the vector into a host cell. In case the host cell is an animal cell, the means mediating entry of the nuclear polypeptide, the polynucleotide, and/or the vector preferably is a transfection reagent; appropriate transfection agents are selected by the skilled person in dependence on the target host cell from known compounds.

Also preferably, the kit further comprises a guide polynucleotide and/or a diluent, preferably further comprises a guide polynucleotide. Appropriate diluents are known to the skilled person and include water, buffers such as phosphate-buffered saline, pharmaceutically compatible diluents, and the like. The term “guide polynucleotide”, in particular “guide RNA”, also referred to as “gRNA”, is known to the skilled person from CRISPR systems, as also described herein above. As used herein, the term guide polynucleotide relates to a polynucleotide conferring sequence specificity to a nuclear polypeptide of the present invention, in particular in case the cargo polypeptide comprised in the nuclear polypeptide is a Cas nuclease or a derivative thereof. Thus, the guide polynucleotide preferably is a gRNA, including a crRNA/tracrRNA hybrid and a crRNA-tracrRNA fusion RNA of a CRISPR/Cas system, as well as a guide RNA of a CPF1 CRISPR system. More preferably, the term gRNA relates to a crRNA-tracrRNA fusion RNA of a CRISPR/Cas system, as well as a guide RNA of a CPF1 CRISPR system. Most preferably, the term gRNA relates to a crRNA-tracrRNA fusion RNA of a CRISPR/Cas system. Preferably, the guide polynucleotide comprises at least 15, preferably at least 18, more preferably at least 20 nucleotides complementary to the target sequence. As used herein, the term “complementary”, if not otherwise noted, relates to at least 90%, more preferably at least 95%, still more preferably 99% complementarity. Most preferably complementarity relates to 100% complementarity over the aforementioned number of nucleotides. Moreover, preferably, the guide polynucleotide of the present invention further comprises a nucleotide sequence mediating binding of a Cpfl endonuclease or comprises tracrRNA sequence, preferably of a CRISPR/Cas system, more preferably of a CRISPR/Cas9 system.

Also preferably, the kit further comprises a means of administration. Means of administration are all means suitable for administering the nuclear polypeptide, the polynucleotide, the vector, and/or the host cell to a subject. The means of administration may include a delivery unit for the administration of the compound or composition and a storage unit for storing said compound or composition until administration. However, it is also contemplated that the means of the current invention may appear as separate devices in such an embodiment and are, preferably, packaged together in said kit. Preferred means for administration are those which can be applied without the particular knowledge of a specialized technician. In a preferred embodiment, the means for administration is a syringe, more preferably with a needle, comprising the compound or composition of the invention. Also preferably, the means for administration is a microinjection device, e.g. a microinjection needle. Also preferably, the means for administration is an intravenous infusion (IV) equipment comprising the compound or composition. In still another preferred embodiment the means for administration is an inhaler comprising the compound of the present invention, wherein, more preferably, said compound is formulated for administration as an aerosol.

Moreover, the present invention relates to a use of a polynucleotide of the present invention, a vector of the present invention, and/or a nuclear polypeptide of the present invention for modifying a polynucleotide comprised in a host cell.

Preferably, said use is an in vitro use; thus, the host cell in the use preferably is a cultured cell, preferably not administered to a subject. Preferably, in the use for modifying a polynucleotide comprised in a host cell, the cargo polypeptide comprised in the nuclear polypeptide is an enzyme acting on at least one polynucleotide as a substrate, more preferably is a nuclease or a base modifying enzyme as specified herein above, still more preferably is a Cas nuclease or a derivative thereof as specified herein above. In accordance, the use preferably is a use of a polynucleotide of the present invention, a vector of the present invention, and/or a nuclear polypeptide of the present invention and, optionally, of a guide polynucleotide for modifying a polynucleotide comprised in a host cell.

The instant invention also relates to a method of treating and/or preventing genetic disease, neurodegenerative disease, cancer, and/or infectious disease, comprising administering a polynucleotide of the present invention, a vector of the present invention, and/or a nuclear polypeptide to an eukaryote, preferably a non-animal eukaryote.

The instant invention also relates to a polynucleotide of the present invention, a vector of the present invention, and/or a nuclear polypeptide for use in medicine, in particular use in treatment and/or prevention of genetic disease, neurodegenerative disease, cancer, and/or infectious disease.

The means and methods of the present invention are, in principle, usable in treatment and/or prevention of each and every disease for which genetic modification of a eukaryotic cell is considered beneficial. In non-animal eukaryotes, preferably in plants, this is the case in particular in genetic disease, cancer, and infectious disease. In animal eukaryotes, such is the case in particular in genetic disease, neurodegenerative disease, cancer, and infectious disease. As used herein, the term “genetic modification”, preferably, includes modification of any kind of nucleic acid comprised in a host cell at a given time, including nuclear DNA, organelle DNA (mitochondrial DNA or plastid DNA), but also nucleic acid from an infectious agent, either as free nucleic acid or covalently connected to the DNA of the host cell. Preferably, genetic modification is modification of nucleic acid, preferably DNA, present in the nucleus of a host cell.

The term “eukaryote” relates to any organism of the domain eukarya, i.e. preferably comprised of one or more eukaryotic cells. Thus, the eukaryote may be a single-cell eukaryotic organism, such as a yeast, an amoeba, or the like. The eukaryote may, however, also be a multicellular organism, such as an animal, a plant, or a fungus. Preferred plants are monocotyledonous or dicotyledonous plants, in particular crop plants. Preferably, the eukaryote is a non-animal eukaryote, i.e. a plant or a fungus. More preferably, the eukaryote is an animal eukaryote, preferably a subject as specified herein below.

The term “subject”, as used herein, relates to an animal, preferably a chordate, more preferably a vertebrate, still more preferably a mammal, an amphibian, or a teleost, the term “mammal” preferably including livestock such as cattle, horse, pig, sheep, and goat, and laboratory animals like a rat, mouse, and guinea pig. Most preferably, the subject is a human. Preferably, livestock and laboratory animals are sacrificed after being used in a method as specified herein.

The term “treatment” refers to an amelioration of the diseases or disorders referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20%, at least 50%, at least 60%, at least 70%, at least 80%, or at least 90% of the subjects of a given cohort or population.

The term “preventing” refers to retaining health with respect to the diseases or disorders referred to herein for a certain period of time in a subject. It will be understood that the said period of time is dependent on a variety of individual factors of the subject and the specific preventive treatment. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms.

Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well known statistic evaluation tools discussed elsewhere in this specification.

The term “genetic disease”, as used herein, relates to a disease or disorder causally linked to one or more modifications, preferably mutations in the genome of an individual. Thus, preferably, the genetic disease is causally linked to one or more epigenetic changes, more preferably is causally linked to one or more genetic mutations. As will be understood, symptoms of a genetic disease often are caused by expression of a mutated gene and/or lack of expression of a gene providing normal function of the gene product in one or more specific tissue(s) and/or cell type(s). Thus, it may be preferable to genetically modify by e.g. Cas activity only those cells in which the mutation contributes to disease. In the case of a non-animal eukaryote, the genetic disease may be an undesirable genetic trait, such as production of off-flavor or off-taste compounds, compounds leading to early spoilage, and the like. In case the eukaryote is a subject, preferably, the genetic disease is Duchenne muscular dystrophy, Huntington's disease, Hemophilia A/B, cystic fibrosis, myotubular myopathy, a glycogen storage disorder, or sickle cell anemia, the causes and symptoms of which are known to the skilled person from textbooks of medicine.

The term “neurodegenerative disease” relates to a disease caused by progressive loss of structure and/or function of neurons in the peripheral and/or central nervous system of an individual. As will be understood, many neurodegenerative diseases are genetic diseases. Preferably, the neurodegenerative disease is a neurodegenerative disease of motoneurons and/or a neurodegenerative disease of the central nervous system. Preferably, the neurodegenerative disease is Alzheimer's disease, amyotrophic lateral sclerosis (ALS), Parkinson's disease, or a spinocerebellar ataxia, preferably spinocerebellar ataxia type 1 (SCA1).

The term “cancer” is, in principle, understood by the skilled person and relates to a disease of an animal or plant, including man, characterized by uncontrolled growth by a group of body cells (“cancer cells”). This uncontrolled growth may be accompanied by intrusion into and destruction of surrounding tissue and possibly spread of cancer cells to other locations in the body (metastasis). Preferably, also included by the term cancer is a relapse. Thus, in case the eukaryote is a subject, preferably, the cancer is a non-solid cancer, e.g. a leukemia, or is a tumor of a solid cancer, a metastasis, or a relapse thereof. As is known to the skilled person, cancer cells accumulate mutations in particular in oncogenes or in tumor-suppressor genes, which may be amenable to correction by genetic modification. Moreover, the means and methods of the present invention may be used to induce cell death, e.g. via apoptosis, specifically in cancer cells. Preferably, treating cancer is reducing tumor and/or cancer cell burden in a subject. As will be understood by the skilled person, effectiveness of treatment of e.g. cancer is dependent on a variety of factors including, e.g. cancer stage and cancer type.

The term “infectious disease” is, in principle, understood by the skilled person. Preferably, the term as used herein relates to an infectious disease in which the replicative cycle of the infectious agent comprises at least one stage in which the genome of the infectious agent is present in a permissive host cell. Thus the infectious disease, preferably, is a viral infection, preferably, in case the eukaryote is a subject, is immunodeficiency virus infection, herpes virus infection, papillomavirus infection, or hepatitis B virus infection.

The present invention also relates to a method for modifying a polynucleotide comprised in a host cell comprising
(a) contacting said host cell with a polynucleotide of the present invention, a vector of the present invention, and/or a nuclear polypeptide of the present invention; and optionally with a guide polynucleotide, and, thereby,
(b) modifying a polynucleotide comprised in said host cell.

The method for modifying a polynucleotide may comprise steps in addition to those explicitly mentioned above. For example, further steps may relate, e.g., to providing a host cell for step a), and/or selecting and/or screening a cell comprising the desired modification of the polynucleotide after step b). Moreover, one or more of said steps may be assisted and/or performed by automated equipment.

The method for modifying a polynucleotide, preferably, is an in vitro method and/or is a non-therapeutic in vivo method; e.g. a method inducing a lethal modification in a polynucleotide in an experimental animal preferably is non-therapeutic. Also, a modification in a polynucleotide having purely cosmetic effects is deemed non-therapeutic, e.g. a modification decreasing pigmentation. The method for modifying a polynucleotide may, however, also be an in vivo method, preferably for treatment and/or prevention of genetic disease, neurodegenerative disease, cancer, and/or infectious disease as specified herein above.

The present invention further relates to a method for determining an in vivo activity of a Cas nuclease comprising (a) contacting a cell with said Cas nuclease and a gRNA causing a gene essential for biosynthesis of a detectable marker to be inactivated; and (b) detecting the detectable marker, and thereby (c) determining in vivo activity of a Cas nuclease.

The method for determining an in vivo activity of a Cas nuclease may comprise steps in addition to those explicitly mentioned above. For example, further steps may relate, e.g., to providing a host cell for step a), and/or, if performed on an experimental animal, sacrificing said experimental animal after step c). Moreover, one or more of said steps may be assisted and/or performed by automated equipment.

The method for determining an in vivo activity of a Cas nuclease is performed in a cultured animal host cell or in an experimental animal. Preferably, the host cell is a host cell, in particular a fertilized oocyte, from zebrafish (Danio rerio) or medaka (Oryzias latipes); preferably, the gRNA is a gRNA targeting the oca2 (oculocutaneous albinism type 2) gene in such case. The method may, however, also be performed in another host cell as specified herein above; in case the host cell does not produce a detectable marker deemed appropriate, e.g. a gene encoding a fluorescent protein, e.g. GFP, may be used as detectable marker; or a gene encoding a detectable enzymatic activity may be used as a target.

In accordance with the above, the present invention also relates to a method for identifying a Cas nuclease with an improved in vivo activity comprising
(a) determining in vivo activity of a candidate Cas nuclease and of a control Cas nuclease according to the method for determining an in vivo activity of a Cas nuclease of the present invention;
(b) comparing the in vivo activity of the candidate Cas nuclease with the in vivo activity of the control Cas nuclease; and
(c) identifying a Cas nuclease with an improved in vivo activity based on the comparison step (b).

The method for identifying a Cas nuclease with an improved in vivo activity may comprise steps in addition to those explicitly mentioned above. For example, further steps may relate, e.g., to providing a host cell for step a), and/or, if performed on an experimental animal, sacrificing said experimental animal after step (c). Moreover, one or more of said steps may be assisted and/or performed by automated equipment.

The term “candidate Cas nuclease”, as used herein, includes any and all Cas nucleases, including variants having a polynucleotide-modifying activity, for which comparison of in vivo activity to a second, non-identical Cas nuclease (the control Cas nuclease) is desirable. In accordance, the term “control Cas nuclease” relates to a Cas nuclease, the activity of which is desirable to be used as a reference point for in vivo activity. As referred to herein, the in vivo activity of the control Cas nuclease does not necessarily have to be known; i.e. preferably, the control Cas nuclease may be a Cas nuclease known to the skilled person to have a high in vitro activity. Preferably, quantification is performed as indicated herein above and/or in the Examples, in particular by determining inactivation of a detectable marker biosynthetic gene, preferably as specified herein above.

The comparison in step (b) may be qualitative, semiquantitative, or quantitative, preferably is quantitative. As is understood by the skilled person, a Cas nuclease with an improved in vivo activity is identified if the in vivo activity of the candidate Cas nuclease is higher, preferably at least 10% higher, more preferably at least 25% higher, still more preferably at least 50% higher, than the control Cas nuclease. Preferably, quantification is performed as indicated herein in the Examples.

The present invention also relates to an N-terminal activity-optimizing peptide (NAO peptide) comprising a tag peptide, a linker peptide, and an NLS peptide, wherein the tag peptide is fused at its C-terminus to the linker peptide.

The components of the NAO peptide have been described herein above. Also preferably, the components of the NAO peptide are directly linked, i.e. there are no amino acids intervening the tag peptide and the linker peptide and/or there are no amino acids intervening the linker peptide and the NLS peptide. Preferably, the sequence of the elements of the NAO peptide (N-terminal to C-terminal) is tag peptide/linker peptide/NLS peptide or NLS peptide/tag peptide/linker peptide; preferably is tag peptide/linker peptide/NLS peptide. Preferably, the NAO peptide comprises, preferably consists of, the amino acid sequence EQKLISEEDLGGSGPPPKRPRLD (SEQ ID NO:11).

In view of the above, the following embodiments are particularly envisaged:

Embodiment 1: A polynucleotide encoding a nuclear polypeptide comprising a cargo polypeptide and an N-terminal activity-optimizing peptide (NAO peptide), wherein said NAO peptide comprises
(i) a tag peptide;
(ii) a linker peptide; and
(iii) a nuclear localization sequence (NLS) peptide;
wherein said linker peptide comprises at least three small amino acids independently selected from glycine, alanine, leucine, serine, aspartate, asparagine, threonine, phenylalanine, glutamate, glutamine, histidine, arginine, lysine, valine, isoleucine, and proline, preferably from glycine, alanine, proline, serine, valine, and threonine; and wherein preferably said tag peptide is fused at its C-terminus to the linker peptide.

Embodiment 2: The polynucleotide of embodiment 1, wherein the sequence of the elements of the NAO peptide is tag peptide/linker peptide/NLS peptide or NLS peptide/tag peptide/linker peptide, preferably is tag peptide/linker peptide/NLS peptide.

Embodiment 3: The polynucleotide of embodiment 1 or 2, wherein said linker peptide comprises said small amino acids within a sequence of at most 10 amino acids, preferably at most 5 amino acids.

Embodiment 4: The polynucleotide of any one of embodiments 1 to 3, wherein said linker peptide comprises at least three consecutive small amino acids.

Embodiment 5: The polynucleotide of any one of embodiments 1 to 4, wherein said small amino acids are independently selected from glycine, alanine, leucine, serine, aspartate, asparagine, threonine, phenylalanine, glutamate, glutamine, histidine, arginine, lysine, valine, isoleucine, and proline, preferably from glycine, alanine, proline, serine, valine, and threonine, preferably from glycine, alanine, proline, and serine, more preferably from glycine and serine.

Embodiment 6: The polynucleotide of any one of embodiments 1 to 5, wherein the linker peptide is a flexible linker peptide comprising, preferably consisting of, the amino acid sequence GG, GS, and/or SG, preferably the amino acid sequence GGS and/or GSG, more preferably the amino acid sequence GGSG (SEQ ID NO:21).

Embodiment 7: The polynucleotide of any one of embodiments 1 to 6, wherein said linker peptide comprises at least three, preferably at least four, consecutive non-charged amino acids. Embodiment 8: The polynucleotide of any one of embodiments 1 to 7, wherein the tag peptide comprises at least two, preferably at least three, more preferably at least four acidic amino acids, preferably within a sequence of ten continuous amino acids.

Embodiment 9: The polynucleotide of any one of embodiments 1 to 8, wherein the tag peptide comprises a c-myc tag peptide, preferably comprises, more preferably consists of, the sequence QKLISEEDL (SEQ ID NO:1) or, more preferably, EQKLISEEDL (SEQ ID NO:2).

Embodiment 10: The polynucleotide of any one of embodiments 1 to 9, wherein said tag peptide fused at its C-terminus to said linker peptide comprises, preferably consists of, the sequence EQKLISEEDLGGSG (SEQ ID NO:3).

Embodiment 11: The polynucleotide of any one of embodiments 1 to 10, wherein said NLS peptide comprises, preferably consists of, the amino acid sequence PPPKRPRLD (SEQ ID NO:4), PKKKRKV (SEQ ID NO:5), RADPKKKRKV (SEQ ID NO:6), RPAATKKAGQAKKKK (SEQ ID NO: 7), APKKKRKVGIHGVPAA (SEQ ID NO:8), PKKKRK (SEQ ID NO:9), APKKKRK (SEQ ID NO:10), or APKKKRKV (SEQ ID NO: 45).

Embodiment 12: The polynucleotide of any one of embodiments 1 to 11, wherein said NLS peptide comprises, preferably consists of, the amino acid sequence PPPKRPRLD (SEQ ID NO:4) or PKKKRKV (SEQ ID NO:5), more preferably PPPKRPRLD (SEQ ID NO:4).

Embodiment 13: The polynucleotide of any one of embodiments 1 to 12, wherein the NAO peptide comprises, preferably consists, of the amino acid sequence EQKLISEEDLGGSGPPPKRPRLD (SEQ ID NO:11).

Embodiment 14: The polynucleotide of any one of embodiments 1 to 13, wherein the cargo polypeptide is (i) an enzyme having at least one of its substrates located in the nucleus of a eukaryotic cell; (ii) a polypeptide having its physiological location in the nucleus of a eukaryotic cell; (iii) any polypeptide for which nuclear localization is desired or desirable; and/or (iv) any polypeptide variant or fragment thereof, wherein a variant or fragment of a polypeptide of (i) or (ii) preferably has the indicated activity and/or amino acid sequence.

Embodiment 15: The polynucleotide of any one of embodiments 1 to 14, wherein said cargo polypeptide is an enzyme having DNA and or RNA as a substrate.

Embodiment 16: The polynucleotide of any one of embodiments 1 to 15, wherein said cargo polypeptide is a nuclease or a base modifying enzyme, preferably a deaminase, a methylase, an acetylase, a transposase, a restriction enzyme, a recombinase, or a DNA-interacting protein.

Embodiment 17: The polynucleotide of any one of embodiments 1 to 16, wherein the cargo polypeptide is a clustered regularly interspaced short palindromic repeats (CRISPR) associated (Cas) polypeptide.

Embodiment 18: The polynucleotide of embodiment 17, wherein the Cas polypeptide is a Cas9 polypeptide.

Embodiment 19: The polynucleotide of embodiment 17 or 18, wherein the Cas polypeptide is a Cas polypeptide from Streptococcus pyogenes or a derivative thereof, preferably a humanized derivative thereof.

Embodiment 20: The polynucleotide of any one of embodiments 17 to 19, wherein the Cas polypeptide is a Cas endonuclease.

Embodiment 21: The polynucleotide of any one of embodiments 17 to 20, wherein said Cas polypeptide has the amino acid sequence of SEQ ID NO:22 or polypeptide variant thereof.

Embodiment 22: The polynucleotide of any one of embodiments 1 to 21, wherein said nuclear polypeptide further comprises an NLS peptide located C-terminally of the cargo polypeptide.

Embodiment 23: The polynucleotide of embodiment 22, wherein said C-terminal NLS peptide has an amino acid sequence as specified in embodiment 11 or 12.

Embodiment 24: The polynucleotide of embodiment 22 or 23, wherein said C-terminal NLS peptide has the same amino acid sequence as the NLS peptide comprised in the NAO peptide, preferably the amino acid sequence of SEQ ID NO:4.

Embodiment 25: The polynucleotide of any one of embodiments 1 to 24, wherein said nuclear polypeptide has the sequence of SEQ ID NO:12, 13, 14, or 15, preferably of SEQ ID NO: 12.

Embodiment 26: The polynucleotide of any one of embodiments 1 to 25, wherein said polynucleotide comprises, preferably consists of, the nucleic acid sequence of SEQ ID NO:16 or a nucleic acid sequence at least 60% identical to SEQ ID NO:16, of SEQ ID NO:17 or a nucleic acid sequence at least 60% identical to SEQ ID NO:17; SEQ ID NO:18 or a nucleic acid sequence at least 60% identical to SEQ ID NO:18, or of SEQ ID NO: 19 or a nucleic acid sequence at least 60% identical to SEQ ID NO:19.

Embodiment 27: A vector comprising a polynucleotide according to any one of embodiments 1 to 26.

Embodiment 28: A polypeptide encoded by a polynucleotide according to any one of embodiments 1 to 26.

Embodiment 29: A host cell comprising a polynucleotide according to any one of embodiments 1 to 26, a vector according to embodiment 27, and/or a nuclear polypeptide according to embodiment 28.

Embodiment 30: A kit comprising a polynucleotide according to any one of embodiments 1 to 26, a vector according to embodiment 27, and/or a polypeptide according to embodiment 28 in a housing.

Embodiment 31: The kit of embodiment 30, further comprising a guide polynucleotide and/or a diluent, preferably further comprising a guide polynucleotide.

Embodiment 32: Use of a polynucleotide according to any one of embodiments 1 to 26, a vector according to embodiment 27, and/or a polypeptide according to embodiment 28 for modifying a polynucleotide comprised in a host cell.

Embodiment 33: A polynucleotide according to any one of embodiments 1 to 26, a vector according to embodiment 27, and/or a polypeptide according to embodiment 28 for use in medicine.

Embodiment 34: A polynucleotide according to any one of embodiments 1 to 26, a vector according to embodiment 27, and/or a polypeptide according to embodiment 28 for use in treating Duchenne muscular dystrophy, Huntington's disease, Hemophilia A/B, cystic fibrosis, myotubular myopathy, a glycogen storage disorder, or sickle cell anemia.

Embodiment 35: A method for modifying a polynucleotide comprised in a host cell comprising
(a) contacting said host cell with a polynucleotide according to any one of 1 to 26, a vector according to embodiment 27, and/or a polypeptide according to embodiment 28; and optionally with a guide polynucleotide, and, thereby,
(b) modifying a polynucleotide comprised in said host cell.

Embodiment 36: The method of embodiment 35, wherein said method is an in vitro method.

Embodiment 37: The method of embodiment 35, wherein said method is a non-therapeutic in vivo method.

Embodiment 38: A method for determining an in vivo activity of a Cas nuclease comprising
(a) contacting a cell with said Cas nuclease and a gRNA causing a gene essential for biosynthesis of a detectable marker to be inactivated; and
(b) detecting the detectable marker, and thereby
(c) determining in vivo activity of a Cas nuclease.

Embodiment 39: The method of embodiment 38, wherein said gene essential for biosynthesis of a detectable marker is a pigment biosynthetic gene.

Embodiment 40: The method of embodiment 38 or 39, wherein said gene essential for biosynthesis of a detectable marker is an albinism gene.

Embodiment 41: The method of any one of embodiments 38 to 40, wherein said cell is a cell from zebrafish (Danio rerio) or medaka (Oryzias latipes), and wherein said gRNA is a gRNA targeting the oca2 (oculocutaneous albinism type 2) gene.

Embodiment 42: A method for identifying a Cas nuclease with an improved in vivo activity comprising
(a) determining in vivo activity of a candidate Cas nuclease and of a control Cas nuclease according to the method according to any one of embodiments 38 to 41;
(b) comparing the in vivo activity of the candidate Cas nuclease with the in vivo activity of the control Cas nuclease; and
(c) identifying a Cas nuclease with an improved in vivo activity based on the comparison step (b).

Embodiment 43: An N-terminal activity-optimizing peptide (NAO peptide) comprising a tag peptide, a linker peptide, and an NLS peptide, wherein the tag peptide is fused at its C-terminus to the linker peptide.

All references cited in this specification are herewith incorporated by reference with respect to their entire disclosure content and the disclosure content specifically mentioned in this specification.

### FIGURE LEGENDS

FIG. 1: heiCas9 exhibits outstanding biallelic targeting activity in fish. Phenotypic range of (1Oca2 T1, T2 and DrOca2 T1, T2 sgRNAs/C′as9 variant mediated loss of pigmentation in medaka (a-d) and zebrafish (e-f). (a) Fully pigmented eyes in uninjected control medaka embryo at 4.5 dpf. (b1-b5) Range of typically observed loss-of-pigmentation phenotypes upon injection with hei('as9 mRNA and OlOca2 T1, T2 sgRNAs. The observed phenotypes range from almost full pigmentation (b1) to completely unpigmented eyes (b5). (c) Minimum intensity projection of a medaka embryo at 4.5 days after injection with heiCas9 and OlOca2 T1, T2 sgRNAs. (d) Locally thresholded pigmentation on elliptical selection per eye (same embryo as in c). (e) Fully pigmented uninjected control zebrafish embryo at 2.5 dpf. (f1-f4) Range of typically observed loss-of-pigmentation phenotypes upon injection with heiCas9 mRNA and DrOca2 T1, T2 sgRNAs. The observed phenotypes range from almost full pigmentation (f1) to completely unpigmented eyes and body (f4).

FIG. 2: heiCas9 (also referred to as MFO-Cas9-O) exhibits the highest bi-allelic targeting activity. (a) Medaka embryos were co-injected with OlOca2 T1, T2 sgRNAs and the mRNAs of different Cas9 variants (for permutation compare pictograms) and analyzed after 4.5 days. (b) Zebrafish embryos were co-injected with DrOca2 T1, T2 sgRNAs and the mRNAs of different Cas9 variants (for permutation compare pictograms) and analyzed after 2.5 days. For each Cas9 variant, pigment loss was quantified yielding mean grey values per eye that ranged from 0, i.e. fully pigmented eyes to 255, i.e. completely unpigmented. I, internal linker; F, flexible linker; M, c-myc tag; n, number of eyes analyzed; O, oNLS; S, SV40 NLS; XI, Xenopus nucleoplasmin bipartite NLS. Statistical analysis was performed in R using pairwise comparisons (Wilcoxon rank sum test, Bonferroni corrected). Medians in (a): uninjected control=0.75; JDS246-Cas9=24.60; myc-Cas9=148.81; heiCas9=196.94. Note: highly significant (p<0.001, as indicated by ***) pigment loss (8-fold increase) in heiCas9 versus JDS246-Cas9 crispants. Medians in (b): uninjected control=2.53; JDS246-Cas9=8.60; heiCas9=234.54. Note: highly significant (p<0.001, as indicated by ***) pigment loss (27-fold increase) in heiCas9 versus JDS246-Cas9 crispants.

FIG. 3: heiCas9 consistently exhibits high genome editing efficiency in mammalian cells. Mouse SW10 cells were co-transfected with MmPrx crRNA and mRNAs of JDS246-Cas9, GeneArt® CRISPR nuclease and heiCas9 respectively. Genome editing efficiency was assessed by TIDE and ICE tools. ICE knock-out score represents proportion of indels that indicate a frameshift or ≥21 bp deletion. Data points represent three biological replicates, black line indicates respective mean: TIDE indel %: JDS246-Cas9=46.2; GeneArt® CRISPR nuclease=46.4, heiCas9=57.1; ICE indel %: JDS246-Cas9=53.3; GeneArt® CRISPR nuclease=54.3, heiCas9=60.3; ICE knock-out score %: JDS246-Cas9=33.7; GeneArt® CRISPR nuclease=35.0, heiCas9=58.3. R2>0.9 (TIDE) and >0.9 (ICE) for all mRNAs tested.

FIG. 4: heiBE4-Gam mediates highly efficient cytosine to thymine transition in medaka embryos. Phenotypic range and quantification of heiBE4-Gam mediated cytosine to thymine transitions in medaka embryos. (a) Categories of typically observed loss-of-pigmentation phenotypes upon injection with BE4-Gam or heiBE4-Gam and OlOca2 TI, T3, T4 sgRNAs. The observed pigmentation phenotypes range from (almost) unpigmented eyes, i.e. a very strong knock-out (top panel; white) over intermediate (central panel; grey) to no loss-of-pigmentation (bottom panel; black). Quantification of phenotype categories resulting from injections with either BE4-Gam or heiBE4-Gam and OlOca2 T1, T3, or T4 sgRNAs. Note: dramatic increase of biallelic knock-out rate when using heiBE4-Gam. n, number of embryos analyzed. (b) Schematic representation of base editing window in OlOca2 TI target site. C-to-T transition of C995 and C996 edits the threonine (T) codon to isoleucine (I) (T3321); C997T creates a pre-mature STOP codon (Q333*). Nucleotide positions refer to open reading frame. (c) Quantification of sanger sequencing reads at nucleotides C995, C996, C997 inside the base editing window of three injected embryo pools reveals overall dramatic increase of C-to-T base transition when using heiBE4-Gam. Note 1.7-fold increase of C997T transition, i.e. efficient introduction of a pre-mature STOP codon. Mean values indicated by thick horizontal lines.

FIG. 5: Targeted mutagenesis with heiCas mediates increased knockout activity and reduced allelic variance compared to standard (zCas9); Illumina sequence analysis of triplicates (eight embryos each) with four different sgRNAs (12.5 ng/μl each) and with reduced Cas mRNA concentration (15 ng/μl zCas9 mRNA (squares), or heiCas9 (circles)); (a) ratio of modified to all Illumina sequences; (b) allelic variance; horizontal lines: mean values; total number of sequences analyzed: OlOca2: zCas9=194931, heiCas9=180222; OlRx2: zCas9=224146, heiCas9=269103; OlRx3: zCas9=195248, heiCas9=175044; OlCryaa: zCas9=209573, heiCas9=200448; statistical evaluation via Student's t-test (R Software).

The following Examples shall merely illustrate the invention. They shall not be construed, whatsoever, to limit the scope of the invention.

### Example 1: Materials and Methods

### 1.1 Fish Maintenance

Zebrafish (Danio rerio) and medaka (Oryzias latipes) fish were bred and maintained as previously described according to standard methods. The animal strains used in the present study were zebrafish AB/back and medaka Cab. All experimental procedures were performed according to the guidelines of the German animal welfare law and approved by the local government (Tierschutzgesetz § 11, Abs. 1, Nr. 1, husbandry permit number 35-9185.64/BH Wittbrodt).

### 1.2 Plasmids

The mammalian codon-optimized (Geneious 8.1.9 (www.geneious.com)) Cas9 sequence was gene-synthesized (GeneArt, ThermoFisher Scientific) as template for cloning heiCas9 using primers (Table 1) containing the sequences coding for the hei-tag (myc-tag (EQKLISEEDL, SEQ ID NO:2), flexible linker (GGSG, SEQ ID NO:21) and an optimized oNLS (PPPKRPRLD, SEQ ID NO:4) (Inuoe, 2016). Cloning into the pCS2+ plasmid (multiple cloning site extended for AgeI site downstream of BamHI site) was performed using AgeI and XbaI restriction sites included in the 5′ region of the forward or reverse primers, respectively. For consistent mRNA synthesis, the published myc-Cas9 (Zhang, 2014) was re-established with the pX330-U6-Chimeric_BB-CBh-hSpCas9 vector as template, primer based exchange of the N-terminal FLAG tag with the myc-tag sequence and brought into pCS2+ using AgeI and XbaI restriction sites included in the 5′ region of the respective primers as well. pX330-U6-Chimeric BB-CBh-hSpCas9 was a gift from Feng Zhang (Addgene plasmid #42230).

BE4-Gam was subcloned from pCMV(BE4-Gam) (Addgene plasmid #100806, a gift from David Liu) in a two step-process, first into pJET1.2 (Thermo Scientific), then into pGGEV4 (Addgene plasmid #49284), by BamHI, EcoRV and KpnI restriction sites to create pGGEV4(BE4-Gam). heiBE4-Gam was assembled into pCS2+ by NEBuilder® HiFi DNA Assembly (NEB) with four inserts using Q5 polymerase PCR products (NEB): pCS2+ backbone, hei-tag, Gam Mu-APOBEC1-Cas9n fragment, Cas9n-UGI fragment, 2xUGI-ONLS (see Table 1 for primers used).

### 1.3 Oligonucleotides

TABLE 1
Primer sequences used: Restriction enzyme sites used for cloning into
pCS2+ plasmid are indicated in italics (AgeI in the forward primer,
XbaI in the reverse primer).

| | | |
|---|---|---|
| Primer name | primer sequence 5′-3′ | SEQ ID NO |

forward

| | | |
|---|---|---|
| myc-Cas9 | AATTTACCGGTCAAACATGGAGCAGAAGCTGATCAG | 23 |
| | CGAGGAGGACCTGATGGCCCCAAAGAAGAAGCGGA | |
| | AGGTC | |
| heiCas9 | AATTTACCGGTTTACCATGGAGCAGAAGCTGATCAG | 24 |
| | CGAGGAGGACCTGGGAGGAAGCGGACCACCTCCCA | |
| | AGAGGCCCAGGCTGGACCTCGAGGATAAAAAGTAT | |
| | TCTATTGGTTTAG | |
| pCS2+ | GCCTCTAGAACTATAGTGAGTCG | 25 |
| backbone | | |
| hei-tag | CTTGTTCTTTTTGCAGGATCCCATTTACCATGGAGCA | 26 |
| | GAAGCTG | |
| Gam Mu- | GACCTCGAGGCTAAACCAGCAAAACGTATCAAG | 27 |
| APOBEC1- | | |
| Cas9n | | |
| fragment | | |
| Cas9n-UGI | GACACTTCTCAAGGCCCTAG | 28 |
| fragment | | |
| 2xUGI-oNLS | CAGCTTGGGGGTGACTCTG | 29 |

reverse

| | | |
|---|---|---|
| heiCas9 | AATTTTCTAGATTAGTCCAGCCTGGGCCTCTTGGGAG | |
| | GAGGGGATCCGTCACCCCCAAGCTGTGAC | 30 |
| myc-Cas9 | AATTTTCTAGATTACTTTTTCTTTTTTGCCTGGCCGGC | 31 |
| pCS2+ | ATGGGATCCTGCAAAAAGAACAAG | 32 |
| backbone | | |
| hei-tag | GCTGGTTTAGCCTCGAGGTCCAGCCTGG | 33 |
| Gam Mu- | CTAGGGCCTTGAGAAGTGTC | 34 |
| APOBEC1- | | |
| Cas9n | | |
| fragment | | |
| Cas9n-UGI | CAGAGTCACCCCCAAGCTG | 35 |
| fragment | | |
| 2xUGI-oNLS | CGACTCACTATAGTTCTAGAGGCTTAGTCCAGCCTG | 36 |
| | GGCCTCTTGGGAGGGGGAGAACCACCAGAGAGC | |

Oca2 sgRNAs for medaka (01Oca2) and zebrafish (DrOca2) were designed using the CCTop target predictor with standard parameters (Stemmer, 2015).

### 1.4 In Vitro Transcription of mRNA

All pCS2+ constructs in this work were linearized using NotI-HF (NEB), pGGEV4(BE4-Gam) was linearized using SpeI-HF (NEB) and mRNA was transcribed in vitro using the mMESSAGE mMACHINE SP6 transcription kit (ThermoFisher Scientific, AM1340). The JDS246-Cas9 was linearized with MssI FD (ThermoFisher Scientific) and transcribed in vitro using the mMESSAGE mMACHINE T7 Ultra Transcription Kit (ThermoFisher Scientific, AM1345). JDS246-Cas9 was a gift from Keith Joung (Addgene plasmid #43861). Oca2 sgRNAs were synthesized using the MEGAscript T7 transcription kit (Thermo Fisher Scientific, AM1334) after plasmid digestion with DraI FD (ThermoFisher Scientific).

### 1.5 Microinjection

Zebrafish and medaka zygotes were injected with ('as9 mRNA at either 30 ng/μl or 150 ng/μl, Oca2 sgRNAs at 30 ng/μl and H2B-GFP mRNA at 10 ng/μl as an injection marker. For the base editing experiments, medaka zygotes were injected with BE4-Gam or heiBE4-Gam mRNA at 50 or 150 ng/μl, Oca2 sgRNA at 30 ng/μl and GFP mRNA at 20 ng/μl as injection tracer. Injected embryos were maintained at 28° C. in zebrafish medium or medaka embryo rearing medium (ERM, 17 mM NaCl; 40 mM KCl; 0.27 mM CaCl₂·2H₂O; 0.66 mM MgSO₄·7H₂O, 17 mM Hepes). Embryos were screened for GFP expression seven hours or one day after injection, and GFP negative specimens were discarded.

### 1.6 Image Acquisition and Phenotype Analysis

Medaka 4.5 days post fertilization (dpf) embryos and zebrafish 2.5 dpf embryos were fixed with 4% paraformaldehyde in 2×PTW (10×PBS pH 7.3, 20% Tween20). Images of medaka embryos were acquired with the high content screening ACQUIFER Imaging Machine (DITABIS AG, Pforzheim, Germany). Images of zebrafish embryos were acquired with a Nikon digital sight DS-Ri1 camera mounted onto a Nikon Microscope SMZ18 and the Nikon Software NIS-Elements F version 4.0. Image analysis was performed with Fiji (Schindelin, 2012) i.e. mean grey values were obtained on minimum intensity projections and locally thresholded (Phansalkar algorithm with parameters r=20, p=0.4, k=0.4) pictures and elliptical selections for each individual eye. The mean grey value per eye was used for the boxplot and statistical analysis (pairwise comparisons using Wilcoxon rank sum test, Bonferroni corrected) in RStudio. For the base editing experiments, pigmentation phenotypes were scored 4.5 days after injection.

### 1.7 Genotyping

Pools of 5 randomly selected embryos were used to prepare DNA for genotypic validation of base editing by lysis in DNA extraction buffer (0.4 M Tris/HCl pH 8.0, 0.15 M NaCl, 0.1% SDS, 5 mM EDTA pH 8.0, 1 mg/ml proteinase K) at 60° C. overnight. Proteinase K was inactivated at 95° C. for 10 min and the solution was diluted 1:2 with H₂O.

Genotyping was performed with Q5 polymerase (NEB), primers Oca2 fwd 5′-GTTAAAACAGTTTCTTAAAAAGAACAGGA-3′ (SEQ ID NO:37) and Oca2 rev 5′-AGCAGAAGAAATGACTCAACATTTTG-3′ (SEQ ID NO:38) (annealing at 62° C.) on 1 μl of diluted DNA sample according to the manufacturer's instructions with 30×PCR cycles. PCR products were analyzed on a 1% agarose gel, bands excised, DNA-extraction performed using innuPREP Gel Extraction Kit (Analytik Jena) according to the manufacturer's instructions and subjected to sequencing.

### 1.8 Cell Lines

Mouse SW10 cells (ATCC, CRL-2766) were cultured in DMEM (Gibco) supplemented with 1 g/ml glucose containing 10% FCS (Sigma), 1% penicillin (10,000 units/ml; Gibco) and 1% streptomycin (10 mg/ml; Gibco) and maintained at 33° C. and 5% CO₂. Cells were passaged at 80-90% confluency. 24 h before transfection cells were seeded in a density of 85,000 cells per 12-well.

### 1.9 CRISPR Transfection

crRNA targeting exon 6 (TCGTATCCAGACACCGTCCC[GGG] (SEQ ID NO:39), PAM in brackets) of the mouse Periaxin (MmPrx) gene was selected from the IDT (crRNA XT) predesign crRNA database. crRNA (50 μM) and Alt-R® CRISPR-Cas9 tracrRNA, ATTO-550 (50 μM; IDT, 1075927) were diluted in nuclease-free duplex buffer (IDT) to a final concentration of 1 μM and incubated at 95° C. for 5 minutes. 1 μg of the corresponding Cas9 mRNA (GeneArt® CRISPR nuclease Invitrogen, A29378; JDS246-Cas9 or heiCas9) and 15 μl of tracrRNA+crRNA Mix (1 μM) were diluted in 34 μl Opti-MEM I (Gibco) and mixed with 3 μl Lipofectamine RNAiMAX (ThermoFisher) diluted in 47 μl Opti-MEM I. The tracrRNA+crRNA lipofection mix was incubated for 20 min at RT. Cell culture medium was exchanged with 900 μl Opti-MEM I and the tracrRNA+crRNA lipofection mix was added dropwise to the well. After 48 h, genomic DNA was extracted using the DNeasy Blood and Tissue Kit (Qiagen, 69506) following the manufacturer's protocol. Q5-PCR was carried out using primers flanking the targeted exon 6 (MmPrx exon 6 fwd 5′-GAGACACTCACTCCAGACCC-3′ (SEQ ID NO:40); MmPrx exon 6 rev 5′-ACTCAGTAACCCAACAGCCA-3′ (SEQ ID NO:41), and 30 cycles. PCR amplicons were purified using the Monarch DNA Gel Extraction Kit (NEB, T1020S) and subjected to sequencing.

### 1.10 Sequencing

Sanger sequencing was performed by Eurofins Genomics using Oca2 fwd 5′-GTTAAAACAGTTTCTTAAAAAGAACAGGA-3′ (SEQ ID NO:37) to evaluate base editing and using MmPrx exon 6 fwd 5′-GAGACACTCACTCCAGACCC-3′ (SEQ ID NO:40) and MmPrx exon 6 rev 5′-ACTCAGTAACCCAACAGCCA-3′ (SEQ ID NO:41) to evaluate genome editing in SW10 cells. Quantification of base editing from sanger sequencing reads was performed with EditR (Kluesner, 2018). Genome editing efficiency was assessed by sequence analysis using the TIDE web tool (Brinkman, 2014) and by ICE (Hsiau, 2019) using default parameters.

### 1.11 Data Visualisation

Data visualisation and figure assembly was performed using Fiji, ggplot2 in RStudio, Geneious Prime 2019.2.1 and Adobe® Illustrator® CS6.

### Example 2: Results

2.1 For nuclear localization of proteins of interest like the Cas9 enzyme, the monopartite NLS originating from the SV40 large T-antigen or a bipartite NLS discovered in the nucleoplasmin of Xenopus are commonly employed. However, during early embryonic development, specific NLSs play a role in differentially regulating nuclear transport arguing for a tight control of their activity. To achieve a high targeting efficiency with a low degree of mosaicism, a high activity should be achieved in the zygote or early cleavage stages. In fish embryos, an optimized artificial NLS (oNLS, Inoue, 2016) facilitates prominent nuclear localization already immediately after fertilization, while the SV40 NLS acts most prominently much later and facilitates nuclear localization approximately at the 1000 cell stage.

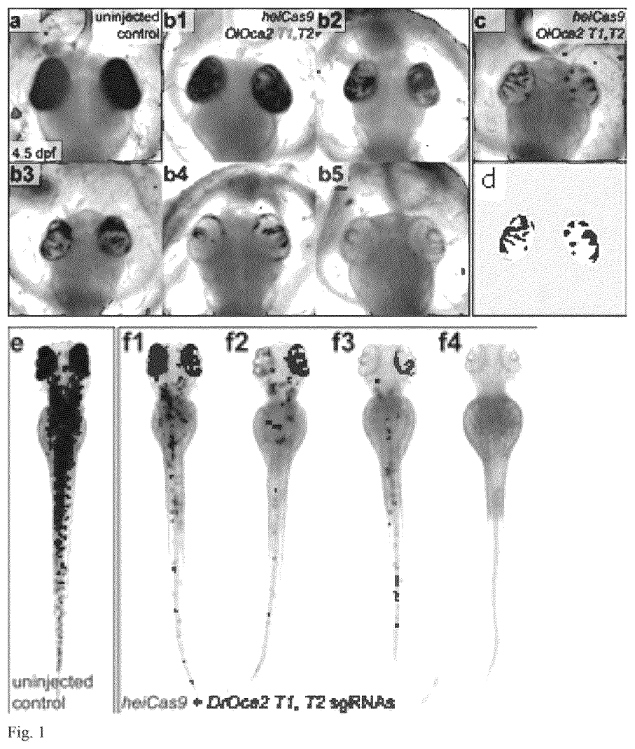

Assessing the efficiency of targeted genome editing requires a reliable and quantitative readout based on an apparent phenotype. We established a quantitative assay for loss-of-eye-pigmentation to address the activity of different Cas9 variants in two teleost model systems, medaka (Oryzias latipes) and zebrafish (Danio rerio) covering a wide evolutionary distance of 200 million years. Our assay on retinal pigmentation provides a clear and reproducible quantitative readout for the loss of the conserved transporter protein oculocutaneous albinism type 2 (oca2), required for melanin biosynthesis (FIG. 1). Only its biallelic inactivation results in the loss of pigmentation of eyes and skin (Lischik, 2019) Altered pigmentation in the eye thus provides a quantitative readout for biallelic targeting efficiency, while the degree of mosaicism is taken as a proxy for the time point of action (uniform-early, mosaic-late).

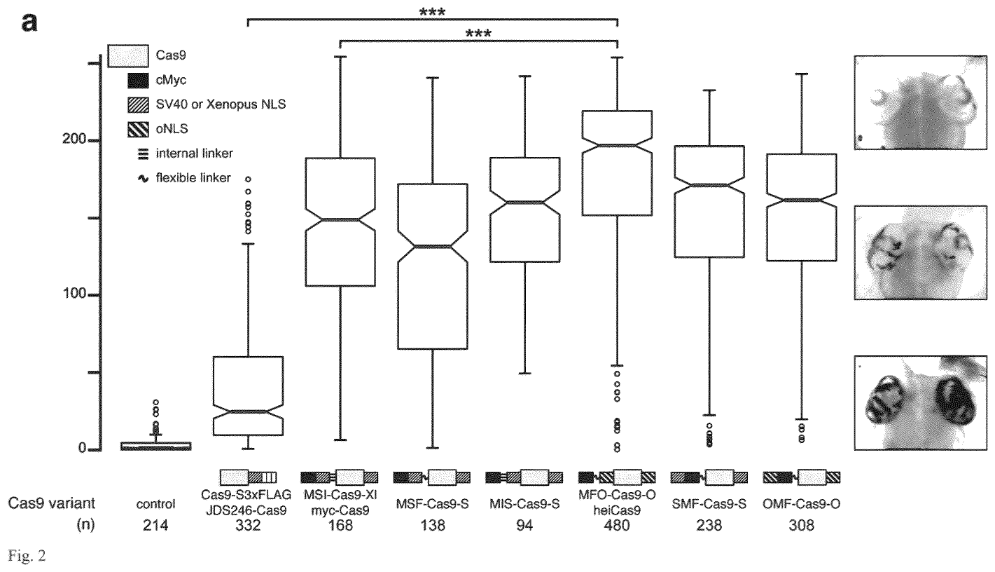

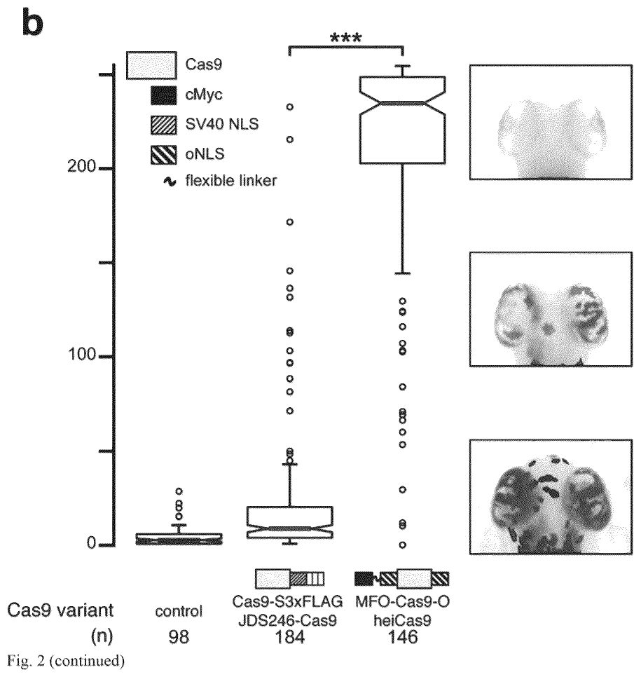

To facilitate uniform Cas9 action, we followed our successful mRNA injection protocol (Gutierrez-Triana, 2018). One-cell stage medaka embryos were co-injected with sgRNAs targeting the oca2 gene (OlOca2 T1, T2) together with mRNA encoding the respective Cas9 variant. Injected embryos were fixed well after the onset of pigmentation at 4.5 days post fertilization and subjected to image analysis (FIG. 1). In brief, the eyes were segmented, (residual) pigmentation was thresholded (FIG. 1) and quantified according to mean grey values (0, i.e. fully pigmented, to 255, i.e. completely unpigmented, FIG. 2).

2.2 We analyzed the activity of the MSI-Cas9-Xl (designated myc-Cas9) variant in which an SV40 NLS (S) and a Xenopus nucleoplasmin bipartite NLS (X1) are flanking the Cas9 enzyme N- and C-terminally, respectively. The N-terminal SV40 NLS is preceded by a c-myc tag (M) and connects to Cas9 via an internal linker (I). This variant showed enhanced activity reflected by the significant increase in mean grey values (median=148.81; FIG. 2a) compared to Cas9-S3xFLAG (designated JDS246-Cas9; median=24.60; FIG. 2a). In addition, we detected reduced mosaicism indicative for targeting activity in earlier cleavage stages. The introduction of two NLSs together with an internal linker as well as the replacement of the C-terminal FLAG tag by a N-terminal c-myc tag resulted in a clear increase in the Cas9 targeting efficiency. We addressed the relative relevance of the individual domains by permutation and replacement with alternative domains. In particular, we compared variants with internal linker or flexible linker (F), SV40 NLS or an optimized NLS (oNLS; O). The oNLS is an importin-alpha dependent proline-rich nona-peptide NLS (PPPKRPRLD, SEQ ID NO:4) displaying a stronger nuclear localization activity compared to the SV40 NLS in early embryonic development in medaka.

We found that for Cas9 variants with SV40 NLS both, positioning or nature of the linker (flexible vs internal) did not significantly impact on the resulting activity of the respective Cas9 variant (compare MSI-Cas9-Xl (median=148.81) with MSF-Cas9-S (median=130.85) and MIS-Cas9-S (median=154.89), FIG. 2a). However, a Cas9 variant N-terminally preceded by a c-myc tag followed by a flexible linker and an optimized NLS (oNLS; O) and C-terminally followed by a second oNLS resulted in the highest targeting activity of the Cas9 variants (MFO-Cas9-O designated heiCas9; median=196.94; FIG. 2a).

2.3 In a second, evolutionarily distant, fish species Danio rerio (zebrafish) we compared the activities of the most extreme Cas9 variants Cas9-S3xFLAG (designated JDS246-Cas9) (median=8.60) and heiCas9 (median=234.54) and, also in zebrafish, found heiCas9 to deliver outstanding targeting efficiency (FIG. 2b).

2.4 We established the base activity level for the assay and addressed the activity of a publicly available Cas9 variant carrying a C-terminal SV40 NLS followed by three FLAG tags (Cas9-S3xFLAG, designated JDS246-Cas9, Plasmid #43861 Addgene, FIG. 2a, b). The analysis of medaka embryos injected with JIS246-Cas9 revealed biallelic inactivation of the oca2 gene as apparent by unpigmented domains in the eye, leading to increased mean grey values (median=24.60) compared to uninjected controls (median=0.75; FIG. 2a). The mosaic distribution of small, unpigmented areas indicated that targeting occurred at later embryonic stages and only in few cells.

2.5 We next analyzed the activity of a myc-Cas9 variant (MSI-Cas9-Xl) in which a SV40 NLS and a Xenopus nucleoplasmin bipartite NLS are flanking the Cas9 enzyme at the N- and C-terminus, respectively (Zhang, 2014). The N-terminal SV40 NLS is preceded by a myc-tag and connects to Cas9 via a VPAA (SEQ ID NO:46) linker, an amino acid spacer regarded as rigid (Huang, 2003). This variant showed a 6-fold enhanced activity as reflected by the increase in mean grey values (median=148.81; FIG. 2a) compared to JDS246-Cas9. In addition, targeting in early embryonic stages was indicated by a lower degree of mosaicism.

2.6 Finally, we addressed the activity of a mammalian codon optimized Cas9 variant featuring a myc-tag, a flexible linker (Huang, 2003) and an oNLS at the N-terminus and a second oNLS at the C-terminus, the combinatorial additions we termed ‘hei-tag’. Embryos co-injected with this “heiCas9” mRNA and sgRNAs against oca2 showed almost no residual pigmentation (median=196.94; FIG. 2 a), proving a significant (p<0.001), 8-fold increase in biallelic targeting efficiency compared to JDS246-Cas9. Using heiCas9 the rate of mosaicism was by far the lowest, arguing for an early time point of action due to high activity and efficient nuclear translocation of the tagged heiCas9 variant already at the earliest cleavage stages.

2.7 To address whether hei-tag fusions to Cas9 variants are widely applicable in different models, we next compared the activities of the JDS246-Cas9 and heiCas9 in Danio rerio (zebrafish) targeting the orthologous oca2 gene (sgRNAs DrOca2 T1, T2). Injected and control embryos were fixed well after the onset of pigmentation at 2.5 dpf (FIG. 1e-f) and subjected to the quantitative assay for eye pigmentation described above. Taking the activity of JDS246-Cas9 as basal level (median=8.60), heiCas9 delivered an outstanding targeting efficiency (median=234.54), reflecting a significant (p<0.001), 27-fold increase (FIG. 2b). Taken together, addition of the hei-tag to a mammalian codon optimized Cas9 resulted in the highly efficient heiCas9, which allows for an up to 27-fold increase in targeting efficiency. It prominently inactivates both alleles of the targeted oca2 locus, with an early onset of action upon injection of heiCas9 mRNA and the respective sgRNAs at the one-cell stage.

2.8 While the early onset of action is required for uniform editing in developing organisms, cell culture approaches demand efficient translocation of the sgRNA/Cas9 complex in a large number of cells. To validate the range of action on the one hand and to address the relevance of the hei-tag in a mammalian setting, we expanded the scope of the analysis to mammalian cell culture. We focused on mRNA-based assays and compared the activity of heiCas9 to state of the art Cas9 variants, i.e. the mRNAs of GeneArt® CRISPR nuclease as well as JDS246-Cas9 in mouse SW10 cells. We assessed the respective genome editing efficiencies by independent and complementary tools, the Tracking of Indels by Decomposition (TIDE) analysis as well as by Inference of CRISPR Editing (ICE). Both approaches decompose the mixed Sanger reads of PCR products spanning the CRISPR target site and compute an efficiency score as well as the distribution of expected indels. Mouse SW10 cells were co-transfected with MmPrx crRNA, an ATTO-550-linked tracrRNA and the mRNAs of either GeneArt® CRISPR nuclease, JDS246-Cas9 or heiCas9. The targeted Periaxin (Prx) locus was PCR amplified and sequenced. Similar to targeting in organismo, heiCas9 also exhibited the highest genome editing efficiency when compared to GeneArt® CRISPR nuclease (TIDE:

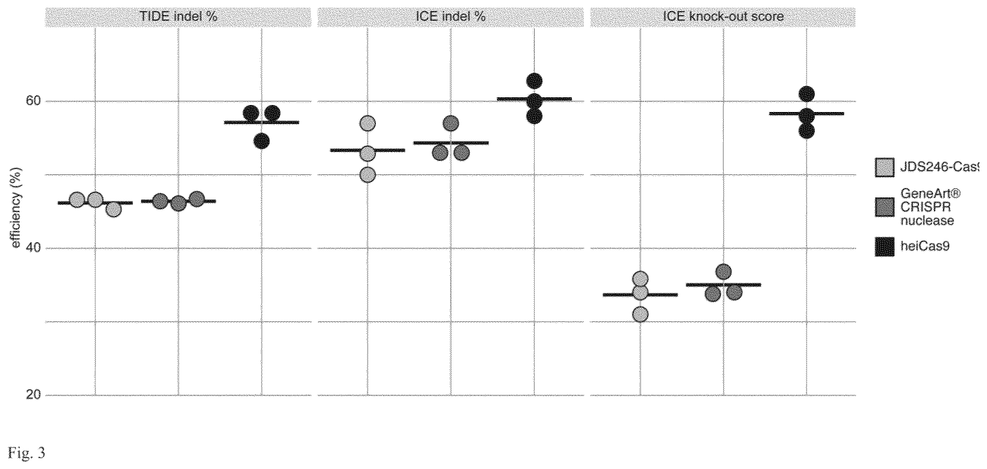

123.1%, ICE: 111%) and JDS246-Cas9 (TIDE: 123.6%, ICE: 113%) in mammalian cell culture (FIG. 3, R²>0.9 (TIDE) and >0.9 (ICE) for all mRNAs tested). Notably, the KO-score efficiencies (ICE) amounted to 167% compared to GeneArt® CRISPR nuclease and to 173% compared to JDS246-Cas9, indicating higher abundance of frameshifts.

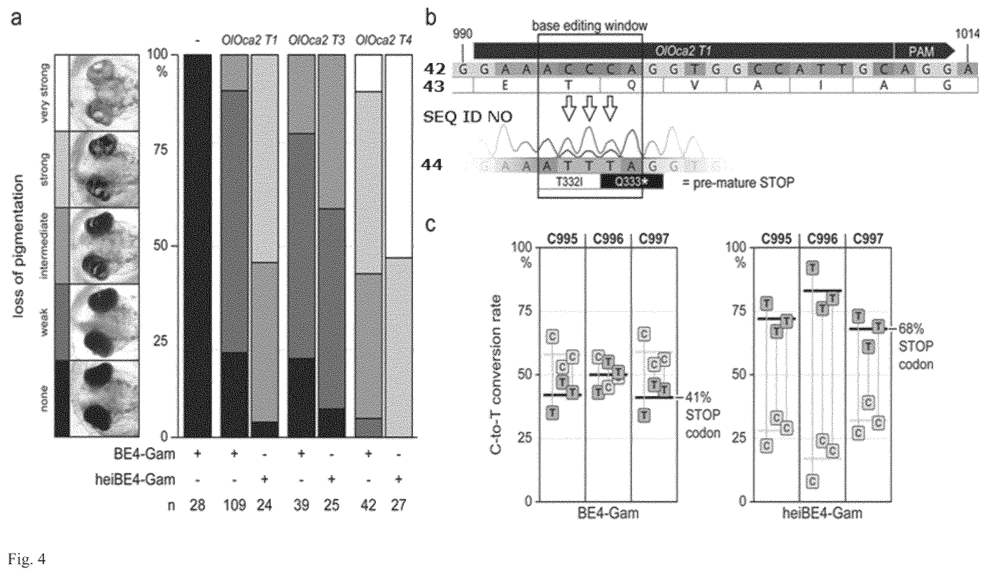

2.9 Given the observed boosting of Cas9 activity by the addition of the hei-tag, we next tested if addition of the hei-tag also improves further Cas9-based techniques. We fused the hei-tag to the C-to-T BE4-Gam base editor (Komor, 2017). In base editors (BEs), a modified Cas9, that does not introduce a DSB (Cas9 nickase or Cas9n) is employed together with a nucleobase deaminase for precisely targeted nucleotide editing. Here, we tested the hei-tag in combination with the C-to-T BE4-Gam base editor (Komor, 2017) to introduce a pre-mature STOP codon in exon 9 of the oca2 open reading frame (ORF) by transition of cytosine 997 to thymine (C997T, leading to Q333*). Again, the loss of pigmentation was used as proxy for biallelic targeting efficiency. Medaka one-cell stage embryos were injected with one of three sgRNAs (OlOca2 T1, T3 or T4) as well as with either BE4-Gam or heiBE4-Gam and screened for pigmentation phenotypes at 4.5 dpf. For each sgRNA, loss of pigmentation was more pronounced when combined with heiBE4-Gam rather than BE4-Gam (FIG. 4a). Quantification of Sanger sequencing reads confirmed a 1.7-fold increase of C997T transition, i.e. introduction of pre-mature STOP codon in case of heiBE4-Gam (FIG. 4b, c). Notably, in heiBE4-Gam injections, for each of the three cytosines in the base editing window, the C-to-T transition rate was higher than 60%, a level never observed in BE4-Gam injected embryos.

### Example 3: Targeted Mutagenesis Efficiency with heiCas9

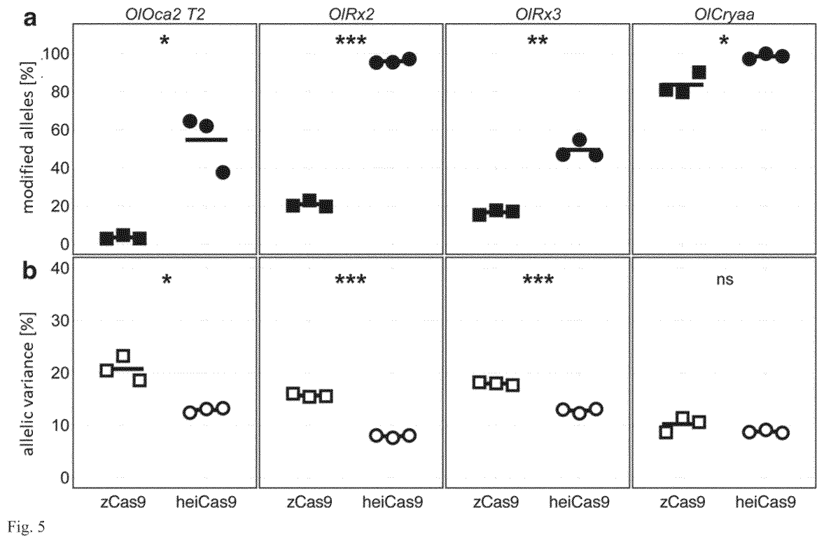

Using an experimental proceeding essentially similar to the preceding Examples, Illumina sequence analysis of triplicates (eight embryos each) was performed with four different sgRNAs (12.5 ng/μl each) and with reduced Cas mRNA concentration (15 ng/μl zCas9 (Jao et al. (2013), PNAS 110(34): 13904) mRNA (squares in FIG. 5), or heiCas9 (circles in FIG. 5). The target sequences of the four sgRNAs lie in different genomic regions: in an exon sequence of the oculocutaneous albinism type 2 (oca2; OlOca2 T2) gene locus, at the start codons of the genes retina-specific homeobox transcription factor 2 (rx2; OlRx2) and alpha a crystallin (cryaa; OlCryaa), and in an intronic sequence of the rx3 (OIRx3) gene locus.

As shown in FIG. 5(a), the ratios of modified to all Illumina sequences in each sample prove a strongly increased knockout-efficiency in the heiCas9 mRNA injections (circles) versus corresponding control (zCas) mRNA injections (squares). The anaylsis of allelic variance in FIG. 5(b), i.e. the mean frequency of each modified allelic sequence in comparison to the number of all modified allelic sequences per relica and gene locus, shows a reduced allelic variance in the heiCas9 injections (empty circles) compared to corresponding control (zCAS9) injections (empty squares).

From the above, it can be concluded that compared to the standard (zCas9), a hei-tag mediates an earlier nuclear localization of Cas9 and an activity in earlier embryonal stages, causing more alleles to be modified with a concomitant rediction of allelic variance. In conclusion from Example 3, heiCas9 has at least the following additional advantages:
increased targeted mutagenesis activity in targeted genome editing;
reduced number of different alleles despite increased mutagenic activity, i.e., without wishing to be bound by theory, earlier nuclear localization and concomitant earlier editing; and
highly efficient editing even at low heiCas9 mRNA concentrations.

### NON-STANDARD REFERENCES

Brinkman, E. K., Chen, T., Amendola, M. & van Steensel, B. Easy quantitative assessment of genome editing by sequence trace decomposition. Nucleic Acids Res 42, e168-e168 (2014).
Cong, L. et al. Multiplex Genome Engineering Using CRISPR/Cas Systems. Science 339, 819-823 (2013).
Gutierrez-Triana, J. A. et al. Efficient single-copy HDR by 5′ modified long dsDNA donors. Elife 7, e39468 (2018).
Hsiau, T. et al. Inference of CRISPR Edits from Sanger Trace Data. Biorxiv 251082 (2019) doi: 10.1101/251082.
Huang, F. & Nau, W. M. A Conformational Flexibility Scale for Amino Acids in Peptides. Angewandte Chemie Int Ed 42, 2269-2272 (2003).
Inoue, T., Iida, A., Maegawa, S., Sehara-Fujisawa, A. & Kinoshita, M. Generation of a transgenic medaka (Oryzias latipes) strain for visualization of nuclear dynamics in early developmental stages. Dev Growth Differ 58, 679-687 (2016).
Jao et al. Efficient multiplex biallelic zebrafish genome editing using a CRISPR nuclease system. PNAS 110(34):13904 (2013).
Kluesner, M. G. et al. EditR: A Method to Quantify Base Editing from Sanger Sequencing. Crispr J 1, 239-250 (2018).
Komor, A. C. et al. Improved base excision repair inhibition and bacteriophage Mu Gam protein yields C:G-to-T:A base editors with higher efficiency and product purity. Sci Adv 3, eaao4774 (2017).
Lischik, C. Q., Adelmann, L. & Wittbrodt, J. Enhanced in vivo-imaging in medaka by optimized anaesthesia, fluorescent protein selection and removal of pigmentation. Plos One 14, e0212956 (2019).
Schindelin, J. et al. Fiji: an open-source platform for biological-image analysis. Nat Methods 9, 676-682 (2012).
Stemmer, M., Thumberger, T., Keyer, M. del S., Wittbrodt, J. & Mateo, J. L. CCTop: An Intuitive, Flexible and Reliable CRISPR/Cas9 Target Prediction Tool. Plos One 10, e0124633 (2015).
US 2014/273226 A1
Wang, H., La Russa, M., Qi, L.S., 2016. CRISPR/Cas9 in Genome Editing and Beyond. Annu. Rev. Biochem. 85, 227-264. doi:10.1146/annurev-biochem-060815-014607
Zhang, J.-H. et al. Improving the specificity and efficacy of CRISPR/CAS9 and gRNA through target specific DNA reporter. J Biotechnol 189, 1-8 (2014).
WO 2014/204727 A1

1. A polynucleotide encoding a nuclear polypeptide comprising a cargo polypeptide and an N-terminal activity-optimizing peptide (NAO peptide), wherein said NAO peptide comprises
(i) a tag peptide;
(ii) a linker peptide; and
(iii) a nuclear localization sequence (NLS) peptide;
wherein said linker peptide comprises at least three small amino acids independently selected from glycine, alanine, leucine, serine, aspartate, asparagine, threonine, phenylalanine, glutamate, glutamine, histidine, arginine, lysine, valine, isoleucine, and proline; and wherein said tag peptide is fused at its C-terminus to the linker peptide.

2. The polynucleotide of claim 1, wherein the linker peptide is a flexible linker peptide comprising the amino acid sequence GG, GS, and/or SG, preferably comprising, more preferably consisting of, the amino acid sequence GGS and/or GSG, more preferably the amino acid sequence GGSG (SEQ ID NO:21).

3. The polynucleotide of claim 1, wherein the tag peptide comprises at least two, preferably at least three, more preferably at least four acidic amino acids, within a sequence of ten continuous amino acids.

4. The polynucleotide of claim 1, wherein the tag peptide comprises a c-myc tag peptide, preferably comprises, more preferably consists of, the sequence QKLISEEDL (SEQ ID NO:1) or, more preferably, EQKLISEEDL (SEQ ID NO:2).

5. The polynucleotide of claim 1, wherein said NLS peptide comprises, preferably consists of, the amino acid sequence PPPKRPRLD (SEQ ID NO:4), PKKKRKV (SEQ ID NO:5), RADPKKKRKV (SEQ ID NO:6), RPAATKKAGQAKKKK (SEQ ID NO: 7), APKKKRKVGIHGVPAA (SEQ ID NO:8), PKKKRK (SEQ ID NO:9), APKKKRK (SEQ ID NO:10), or APKKKRKV (SEQ ID NO: 45).

6. The polynucleotide of claim 1, wherein the NAO peptide comprises, preferably consists, of the amino acid sequence EQKLISEEDLGGSGPPPKRPRLD (SEQ ID NO:11).

7. The polynucleotide of claim 1, wherein the linker peptide is a flexible linker peptide comprising the amino acid sequence GG, GS, and/or SG and wherein the tag peptide comprises a c-myc tag peptide.

8. The polynucleotide of claim 1, wherein the linker peptide is a flexible linker peptide comprising the amino acid sequence GG, GS, and/or SG and wherein said NLS peptide comprises, preferably consists of, the amino acid sequence PPPKRPRLD (SEQ ID NO:4), PKKKRKV (SEQ ID NO:5), RADPKKKRKV (SEQ ID NO:6), RPAATKKAGQAKKKK (SEQ ID NO: 7), APKKKRKVGIHGVPAA (SEQ ID NO:8), PKKKRK (SEQ ID NO:9), APKKKRK (SEQ ID NO:10), or APKKKRKV (SEQ ID NO: 45).

9. The polynucleotide of claim 1, wherein the linker peptide is a flexible linker peptide comprising the amino acid sequence GG, GS, and/or SG, wherein the tag peptide comprises a c-myc tag peptide, and wherein said NLS peptide comprises, preferably consists of, the amino acid sequence PPPKRPRLD (SEQ ID NO:4), PKKKRKV (SEQ ID NO:5), RADPKKKRKV (SEQ ID NO:6), RPAATKKAGQAKKKK (SEQ ID NO: 7), APKKKRKVGIHGVPAA (SEQ ID NO:8), PKKKRK (SEQ ID NO:9), APKKKRK (SEQ ID NO:10), or APKKKRKV (SEQ ID NO: 45).

10. The polynucleotide of claim 1, wherein said cargo polypeptide is an enzyme having DNA and or RNA as a substrate.

11. The polynucleotide of claim 1, wherein said cargo polypeptide is a nuclease or a base modifying enzyme, preferably a deaminase, a methylase, an acetylase, a transposase, a restriction enzyme, a recombinase, or a DNA-interacting protein; preferably wherein the cargo polypeptide is a clustered regularly interspaced short palindromic repeats (CRISPR) associated (Cas) polypeptide.

12. The polynucleotide of claim 1, wherein said nuclear polypeptide further comprises an NLS peptide located C-terminally of the cargo polypeptide.

13. The polynucleotide of claim 1, wherein said nuclear polypeptide has the sequence of SEQ ID NO:12, 13, 14, or 15, preferably of SEQ ID NO:12.

14. The polynucleotide of claim 1, wherein said polynucleotide comprises, preferably consists of, the nucleic acid sequence of SEQ ID NO:16 or a nucleic acid sequence at least 60% identical to SEQ ID NO:16, of SEQ ID NO:17 or a nucleic acid sequence at least 60% identical to SEQ ID NO:17; SEQ ID NO:18 or a nucleic acid sequence at least 60% identical to SEQ ID NO:18, or of SEQ ID NO:19 or a nucleic acid sequence at least 60% identical to SEQ ID NO: 19.

15. A polypeptide encoded by a polynucleotide according to claim 1.

16. (canceled).

17. (canceled).

18. (canceled).

19. A method of treating and/or preventing genetic disease, neurodegenerative disease, cancer, and/or infectious disease, comprising administering a polynucleotide according to claim 1 to a eukaryote.

20. The method of claim 19, wherein said eukaryote is a vertebrate.

21. The method of claim 19, wherein said eukaryote is a human.

22. The method of claim 19, wherein said genetic disease is Duchenne muscular dystrophy, Huntington's disease, Hemophilia A/B, cystic fibrosis, myotubular myopathy, a glycogen storage disorder, or sickle cell anemia.
